SEQUENCE LISTING (155 sequences)

SEQ ID NO: 1 - protein, 4 aa, synthetic construct
FFFF

SEQ ID NO: 2 - protein, 4 aa, synthetic construct
FFKF

SEQ ID NO: 3 - protein, 4 aa, synthetic construct
FFKY

SEQ ID NO: 4 - protein, 4 aa, synthetic construct
FFYK

SEQ ID NO: 5 - protein, 4 aa, synthetic construct
FFFK

SEQ ID NO: 6 - protein, 5 aa, synthetic construct
FFFFF

SEQ ID NO: 7 - protein, 5 aa, synthetic construct
FFGFF

SEQ ID NO: 8 - protein, 5 aa, synthetic construct
FFFGF

SEQ ID NO: 9 - protein, 5 aa, synthetic construct
FFFFG

SEQ ID NO: 10 - protein, 4 aa, synthetic construct
FFFE

SEQ ID NO: 11 - protein, 4 aa, synthetic construct
FFKE

SEQ ID NO: 12 - protein, 4 aa, synthetic construct
FFEK

SEQ ID NO: 13 - protein, 5 aa, synthetic construct
FFFFE

SEQ ID NO: 14 - protein, 5 aa, synthetic construct
FFEFF

SEQ ID NO: 15 - protein, 5 aa, synthetic construct
FFFEF

SEQ ID NO: 16 - protein, 4 aa, synthetic construct
FFFD

SEQ ID NO: 17 - protein, 4 aa, synthetic construct
FFKD

SEQ ID NO: 18 - protein, 4 aa, synthetic construct
FFDK

SEQ ID NO: 19 - protein, 5 aa, synthetic construct
FFFFD

SEQ ID NO: 20 - protein, 5 aa, synthetic construct
FFDFF

SEQ ID NO: 21 - protein, 5 aa, synthetic construct
FFFDF

SEQ ID NO: 22 - skipped

SEQ ID NO: 23 - protein, 5 aa, synthetic construct
  VARIANT 1: note = where X is 3-(1-Naphthyl)-D-alanine, 3-(2-Naphthyl)-D-alanine), (3-(1-Naphthyl)-L-alanine or 3-(2-Naphthyl)-L-alanine
XFFFF

SEQ ID NO: 24 - protein, 5 aa, synthetic construct
  VARIANT 1: note = where X is 3-(1-Naphthyl)-D-alanine, 3-(2-Naphthyl)-D-alanine), (3-(1-Naphthyl)-L-alanine or 3-(2-Naphthyl)-L-alanine
XFFKF

SEQ ID NO: 25 - protein, 5 aa, synthetic construct
  VARIANT 1: note = where X is 3-(1-Naphthyl)-D-alanine, 3-(2-Naphthyl)-D-alanine), (3-(1-Naphthyl)-L-alanine or 3-(2-Naphthyl)-L-alanine
XFFKY

SEQ ID NO: 26 - protein, 5 aa, synthetic construct
  VARIANT 1: note = where X is 3-(1-Naphthyl)-D-alanine, 3-(2-Naphthyl)-D-alanine), (3-(1-Naphthyl)-L-alanine or 3-(2-Naphthyl)-L-alanine
XFFYK

SEQ ID NO: 27 - protein, 5 aa, synthetic construct
  VARIANT 1: note = where X is 3-(1-Naphthyl)-D-alanine, 3-(2-Naphthyl)-D-alanine), (3-(1-Naphthyl)-L-alanine or 3-(2-Naphthyl)-L-alanine
XFFFK

SEQ ID NO: 28 - protein, 6 aa, synthetic construct
  VARIANT 1: note = where X is 3-(1-Naphthyl)-D-alanine, 3-(2-Naphthyl)-D-alanine), (3-(1-Naphthyl)-L-alanine or 3-(2-Naphthyl)-L-alanine
XFFFFF

SEQ ID NO: 29 - protein, 6 aa, synthetic construct
  VARIANT 1: note = where X is 3-(1-Naphthyl)-D-alanine, 3-(2-Naphthyl)-D-alanine), (3-(1-Naphthyl)-L-alanine or 3-(2-Naphthyl)-L-alanine
XFFGFF

SEQ ID NO: 30 - protein, 6 aa, synthetic construct
  VARIANT 1: note = where X is 3-(1-Naphthyl)-D-alanine, 3-(2-Naphthyl)-D-alanine), (3-(1-Naphthyl)-L-alanine or 3-(2-Naphthyl)-L-alanine
XFFFGF

SEQ ID NO: 31 - protein, 6 aa, synthetic construct
  VARIANT 1: note = where X is 3-(1-Naphthyl)-D-alanine, 3-(2-Naphthyl)-D-alanine), (3-(1-Naphthyl)-L-alanine or 3-(2-Naphthyl)-L-alanine
XFFFFG

SEQ ID NO: 32 - skipped

SEQ ID NO: 33 - protein, 5 aa, synthetic construct
  VARIANT 1: note = where X is 3-(1-Naphthyl)-D-alanine, 3-(2-Naphthyl)-D-alanine), (3-(1-Naphthyl)-L-alanine or 3-(2-Naphthyl)-L-alanine
XFFFE

SEQ ID NO: 34 - protein, 5 aa, synthetic construct
  VARIANT 1: note = where X is 3-(1-Naphthyl)-D-alanine, 3-(2-Naphthyl)-D-alanine), (3-(1-Naphthyl)-L-alanine or 3-(2-Naphthyl)-L-alanine
XFFKE

SEQ ID NO: 35 - protein, 5 aa, synthetic construct
  VARIANT 1: note = where X is 3-(1-Naphthyl)-D-alanine, 3-(2-Naphthyl)-D-alanine), (3-(1-Naphthyl)-L-alanine or 3-(2-Naphthyl)-L-alanine
XFFEK

SEQ ID NO: 36 - protein, 6 aa, synthetic construct
  VARIANT 1: note = where X is 3-(1-Naphthyl)-D-alanine, 3-(2-Naphthyl)-D-alanine), (3-(1-Naphthyl)-L-alanine or 3-(2-Naphthyl)-L-alanine
XFFFFE

SEQ ID NO: 37 - protein, 6 aa, synthetic construct
  VARIANT 1: note = where X is 3-(1-Naphthyl)-D-alanine, 3-(2-Naphthyl)-D-alanine), (3-(1-Naphthyl)-L-alanine or 3-(2-Naphthyl)-L-alanine
XFFEFF

SEQ ID NO: 38 - protein, 6 aa, synthetic construct
  VARIANT 1: note = where X is 3-(1-Naphthyl)-D-alanine, 3-(2-Naphthyl)-D-alanine), (3-(1-Naphthyl)-L-alanine or 3-(2-Naphthyl)-L-alanine
XFFFEF

SEQ ID NO: 39 - skipped

SEQ ID NO: 40 - protein, 5 aa, synthetic construct
  VARIANT 1: note = where X is 3-(1-Naphthyl)-D-alanine, 3-(2-Naphthyl)-D-alanine), (3-(1-Naphthyl)-L-alanine or 3-(2-Naphthyl)-L-alanine
XFFFD

SEQ ID NO: 41 - protein, 5 aa, synthetic construct
  VARIANT 1: note = where X is 3-(1-Naphthyl)-D-alanine, 3-(2-Naphthyl)-D-alanine), (3-(1-Naphthyl)-L-alanine or 3-(2-Naphthyl)-L-alanine
XFFKD

SEQ ID NO: 42 - protein, 5 aa, synthetic construct
  VARIANT 1: note = where X is 3-(1-Naphthyl)-D-alanine, 3-(2-Naphthyl)-D-alanine), (3-(1-Naphthyl)-L-alanine or 3-(2-Naphthyl)-L-alanine
XFFDK

SEQ ID NO: 43 - protein, 6 aa, synthetic construct
  VARIANT 1: note = where X is 3-(1-Naphthyl)-D-alanine, 3-(2-Naphthyl)-D-alanine), (3-(1-Naphthyl)-L-alanine or 3-(2-Naphthyl)-L-alanine
XFFFFD

SEQ ID NO: 44 - protein, 6 aa, synthetic construct
  VARIANT 1: note = where X is 3-(1-Naphthyl)-D-alanine, 3-(2-Naphthyl)-D-alanine), (3-(1-Naphthyl)-L-alanine or 3-(2-Naphthyl)-L-alanine
XFFDFF

SEQ ID NO: 45 - protein, 6 aa, synthetic construct
  VARIANT 1: note = where X is 3-(1-Naphthyl)-D-alanine, 3-(2-Naphthyl)-D-alanine), (3-(1-Naphthyl)-L-alanine or 3-(2-Naphthyl)-L-alanine
XFFFDF

SEQ ID NO: 46 - protein, 6 aa, synthetic construct
  VARIANT 1: note = where X at position 1 is a phosphorylated aryl group
  VARIANT 6: note = X at position 6 is optional and can be an O-methyl group, a fluorophore, a chemotherapeutic agent, an antiangiogenic agent, a thermoablative nanoparticle, an immunomodulating agent, or an antigen
XFFFFX

SEQ ID NO: 47 - protein, 6 aa, synthetic construct
  VARIANT 1: note = where X at position 1 is a phosphorylated aryl group
  VARIANT 6: note = X at position 6 is optional and can be an O-methyl group, a fluorophore, a chemotherapeutic agent, an antiangiogenic agent, a thermoablative nanoparticle, an immunomodulating agent, or an antigen
XFFKFX

SEQ ID NO: 48 - protein, 6 aa, synthetic construct
  VARIANT 1: note = where X at position 1 is a phosphorylated aryl group
  VARIANT 6: note = X at position 6 is optional and can be an O-methyl group, a fluorophore, a chemotherapeutic agent, an antiangiogenic agent, a thermoablative nanoparticle, an immunomodulating agent, or an antigen
XFFKYX

SEQ ID NO: 49 - protein, 6 aa, synthetic construct
  VARIANT 1: note = where X at position 1 is a phosphorylated aryl group
  VARIANT 6: note = X at position 6 is optional and can be an O-methyl group, a fluorophore, a chemotherapeutic agent, an antiangiogenic agent, a thermoablative nanoparticle, an immunomodulating agent, or an antigen
XFFYKX

SEQ ID NO: 50 - protein, 6 aa, synthetic construct
  VARIANT 1: note = where X at position 1 is a phosphorylated aryl group
  VARIANT 6: note = X at position 6 is optional and can be an O-methyl group, a fluorophore, a chemotherapeutic agent, an antiangiogenic agent, a thermoablative nanoparticle, an immunomodulating agent, or an antigen
XFFFKX

SEQ ID NO: 51 - protein, 7 aa, synthetic construct
  VARIANT 1: note = where X at position 1 is a phosphorylated aryl group
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XFFFFFX

SEQ ID NO: 52 - protein, 7 aa, synthetic construct
  VARIANT 1: note = where X at position 1 is a phosphorylated aryl group
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XFFGFFX

SEQ ID NO: 53 - protein, 7 aa, synthetic construct
  VARIANT 1: note = where X at position 1 is a phosphorylated aryl group
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XFFFGFX

SEQ ID NO: 54 - protein, 7 aa, synthetic construct
  VARIANT 1: note = where X at position 1 is a phosphorylated aryl group
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XFFFFGX

SEQ ID NO: 55-62 - skipped (8 entries)

SEQ ID NO: 63 - protein, 7 aa, synthetic construct
  VARIANT 1: note = where X at position 1 is a phosphorylated aryl group
  MOD_RES 6: note = X at position 6 is glutamic acid or glutamic acid having its sidechain modified with an O-methyl group
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XFFFFXX

SEQ ID NO: 64 - protein, 7 aa, synthetic construct
  VARIANT 1: note = where X at position 1 is a phosphorylated aryl group
  MOD_RES 4: note = X at position 4 is glutamic acid or glutamic acid having its sidechain modified with an O-methyl group
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XFFXFFX

SEQ ID NO: 65 - protein, 7 aa, synthetic construct
  VARIANT 1: note = where X at position 1 is a phosphorylated aryl group
  MOD_RES 5: note = X at position 5 is glutamic acid or glutamic acid having its sidechain modified with an O-methyl group
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XFFFXFX

SEQ ID NO: 66-69 - skipped (4 entries)

SEQ ID NO: 70 - protein, 7 aa, synthetic construct
  VARIANT 1: note = where X at position 1 is a phosphorylated aryl group
  MOD_RES 6: note = X at position 6 is aspartic acid or aspartic acid having its sidechain modified with an O-methyl group
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XFFFFXX

SEQ ID NO: 71 - protein, 7 aa, synthetic construct
  VARIANT 1: note = where X at position 1 is a phosphorylated aryl group
  MOD_RES 4: note = X at position 4 is aspartic acid or aspartic acid having its sidechain modified with an O-methyl group
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XFFXFFX

SEQ ID NO: 72 - protein, 7 aa, synthetic construct
  VARIANT 1: note = where X at position 1 is a phosphorylated aryl group
  MOD_RES 5: note = X at position 5 is aspartic acid or aspartic acid having its sidechain modified with an O-methyl group
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XFFFXFX

SEQ ID NO: 73 - skipped

SEQ ID NO: 74 - protein, 7 aa, synthetic construct
  VARIANT 1: note = where X at position 1 is a phosphorylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XXFFFFX

SEQ ID NO: 75 - protein, 7 aa, synthetic construct
  VARIANT 1: note = where X at position 1 is a phosphorylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XXFFKFX

SEQ ID NO: 76 - protein, 7 aa, synthetic construct
  VARIANT 1: note = where X at position 1 is a phosphorylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XXFFKYX

SEQ ID NO: 77 - protein, 7 aa, synthetic construct
  VARIANT 1: note = where X at position 1 is a phosphorylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XXFFYKX

SEQ ID NO: 78 - protein, 7 aa, synthetic construct
  VARIANT 1: note = where X at position 1 is a phosphorylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XXFFFKX

SEQ ID NO: 79 - protein, 8 aa, synthetic construct
  VARIANT 1: note = where X at position 1 is a phosphorylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  VARIANT 8: note = X at position 8 is optional and can be an O-methyl group, a fluorophore, a chemotherapeutic agent, an antiangiogenic agent, a thermoablative nanoparticle, an immunomodulating agent, or an antigen
XXFFFFFX

SEQ ID NO: 80 - protein, 8 aa, synthetic construct
  VARIANT 1: note = where X at position 1 is a phosphorylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  VARIANT 8: note = X at position 8 is optional and can be an O-methyl group, a fluorophore, a chemotherapeutic agent, an antiangiogenic agent, a thermoablative nanoparticle, an immunomodulating agent, or an antigen
XXFFGFFX

SEQ ID NO: 81 - protein, 8 aa, synthetic construct
  VARIANT 1: note = where X at position 1 is a phosphorylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  VARIANT 8: note = X at position 8 is optional and can be an O-methyl group, a fluorophore, a chemotherapeutic agent, an antiangiogenic agent, a thermoablative nanoparticle, an immunomodulating agent, or an antigen
XXFFFGFX

SEQ ID NO: 82 - protein, 8 aa, synthetic construct
  VARIANT 1: note = where X at position 1 is a phosphorylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  VARIANT 8: note = X at position 8 is optional and can be an O-methyl group, a fluorophore, a chemotherapeutic agent, an antiangiogenic agent, a thermoablative nanoparticle, an immunomodulating agent, or an antigen
XXFFFFGX

SEQ ID NO: 83-90 - skipped (8 entries)

SEQ ID NO: 91 - protein, 8 aa, synthetic construct
  VARIANT 1: note = where X at position 1 is a phosphorylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  MOD_RES 7: note = X at position 7 is glutamic acid or glutamic acid having its sidechain modified with an O-methyl group
  VARIANT 8: note = X at position 8 is optional and can be an O-methyl group, a fluorophore, a chemotherapeutic agent, an antiangiogenic agent, a thermoablative nanoparticle, an immunomodulating agent, or an antigen
XXFFFFXX

SEQ ID NO: 92 - protein, 8 aa, synthetic construct
  VARIANT 1: note = where X at position 1 is a phosphorylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  MOD_RES 5: note = X at position 5 is glutamic acid or glutamic acid having its sidechain modified with an O-methyl group
  VARIANT 8: note = X at position 8 is optional and can be an O-methyl group, a fluorophore, a chemotherapeutic agent, an antiangiogenic agent, a thermoablative nanoparticle, an immunomodulating agent, or an antigen
XXFFXFFX

SEQ ID NO: 93 - protein, 8 aa, synthetic construct
  VARIANT 1: note = where X at position 1 is a phosphorylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  MOD_RES 6: note = X at position 6 is glutamic acid or glutamic acid having its sidechain modified with an O-methyl group
  VARIANT 8: note = X at position 8 is optional and can be an O-methyl group, a fluorophore, a chemotherapeutic agent, an antiangiogenic agent, a thermoablative nanoparticle, an immunomodulating agent, or an antigen
XXFFFXFX

SEQ ID NO: 94-97 - skipped (4 entries)

SEQ ID NO: 98 - protein, 8 aa, synthetic construct
  VARIANT 1: note = where X at position 1 is a phosphorylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  MOD_RES 7: note = X at position 7 is aspartic acid or aspartic acid having its sidechain modified with an O-methyl group
  VARIANT 8: note = X at position 8 is optional and can be an O-methyl group, a fluorophore, a chemotherapeutic agent, an antiangiogenic agent, a thermoablative nanoparticle, an immunomodulating agent, or an antigen
XXFFFFXX

SEQ ID NO: 99 - protein, 8 aa, synthetic construct
  VARIANT 1: note = where X at position 1 is a phosphorylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  MOD_RES 5: note = X at position 5 is aspartic acid or aspartic acid having its sidechain modified with an O-methyl group
  VARIANT 8: note = X at position 8 is optional and can be an O-methyl group, a fluorophore, a chemotherapeutic agent, an antiangiogenic agent, a thermoablative nanoparticle, an immunomodulating agent, or an antigen
XXFFXFFX

SEQ ID NO: 100 - protein, 8 aa, synthetic construct
  VARIANT 1: note = where X at position 1 is a phosphorylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  MOD_RES 6: note = X at position 6 is aspartic acid or aspartic acid having its sidechain modified with an O-methyl group
  VARIANT 8: note = X at position 8 is optional and can be an O-methyl group, a fluorophore, a chemotherapeutic agent, an antiangiogenic agent, a thermoablative nanoparticle, an immunomodulating agent, or an antigen
XXFFFXFX

SEQ ID NO: 101 - protein, 6 aa, synthetic construct
  VARIANT 1: note = where X at position 1 is a hydroxylated aryl group
  VARIANT 6: note = X at position 6 is optional and can be an O-methyl group, a fluorophore, a chemotherapeutic agent, an antiangiogenic agent, a thermoablative nanoparticle, an immunomodulating agent, or an antigen
XFFFFX

SEQ ID NO: 102 - protein, 6 aa, synthetic construct
  VARIANT 1: note = where X at position 1 is a hydroxylated aryl group
  VARIANT 6: note = X at position 6 is optional and can be an O-methyl group, a fluorophore, a chemotherapeutic agent, an antiangiogenic agent, a thermoablative nanoparticle, an immunomodulating agent, or an antigen
XFFKFX

SEQ ID NO: 103 - protein, 6 aa, synthetic construct
  VARIANT 1: note = where X at position 1 is a hydroxylated aryl group
  VARIANT 6: note = X at position 6 is optional and can be an O-methyl group, a fluorophore, a chemotherapeutic agent, an antiangiogenic agent, a thermoablative nanoparticle, an immunomodulating agent, or an antigen
XFFKYX

SEQ ID NO: 104 - protein, 6 aa, synthetic construct
  VARIANT 1: note = where X at position 1 is a hydroxylated aryl group
  VARIANT 6: note = X at position 6 is optional and can be an O-methyl group, a fluorophore, a chemotherapeutic agent, an antiangiogenic agent, a thermoablative nanoparticle, an immunomodulating agent, or an antigen
XFFYKX

SEQ ID NO: 105 - protein, 6 aa, synthetic construct
  VARIANT 1: note = where X at position 1 is a hydroxylated aryl group
  VARIANT 6: note = X at position 6 is optional and can be an O-methyl group, a fluorophore, a chemotherapeutic agent, an antiangiogenic agent, a thermoablative nanoparticle, an immunomodulating agent, or an antigen
XFFFKX

SEQ ID NO: 106 - protein, 7 aa, synthetic construct
  VARIANT 1: note = where X at position 1 is a hydroxylated aryl group
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XFFFFFX

SEQ ID NO: 107 - protein, 7 aa, synthetic construct
  VARIANT 1: note = where X at position 1 is a hydroxylated aryl group
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XFFGFFX

SEQ ID NO: 108 - protein, 7 aa, synthetic construct
  VARIANT 1: note = where X at position 1 is a hydroxylated aryl group
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XFFFGFX

SEQ ID NO: 109 - protein, 7 aa, synthetic construct
  VARIANT 1: note = where X at position 1 is a hydroxylated aryl group
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XFFFFGX

SEQ ID NO: 110-117 - skipped (8 entries)

SEQ ID NO: 118 - protein, 7 aa, synthetic construct
  VARIANT 1: note = where X at position 1 is a hydroxylated aryl group
  MOD_RES 6: note = X at position 6 is glutamic acid or glutamic acid having its sidechain modified with an O-methyl group
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XFFFFXX

SEQ ID NO: 119 - protein, 7 aa, synthetic construct
  VARIANT 1: note = where X at position 1 is a hydroxylated aryl group
  MOD_RES 4: note = X at position 4 is glutamic acid or glutamic acid having its sidechain modified with an O-methyl group
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XFFXFFX

SEQ ID NO: 120 - protein, 7 aa, synthetic construct
  VARIANT 1: note = where X at position 1 is a hydroxylated aryl group
  MOD_RES 5: note = X at position 5 is glutamic acid or glutamic acid having its sidechain modified with an O-methyl group
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XFFFXFX

SEQ ID NO: 121-124 - skipped (4 entries)

SEQ ID NO: 125 - protein, 7 aa, synthetic construct
  VARIANT 1: note = where X at position 1 is a hydroxylated aryl group
  MOD_RES 6: note = X at position 6 is aspartic acid or aspartic acid having its sidechain modified with an O-methyl group
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XFFFFXX

SEQ ID NO: 126 - protein, 7 aa, synthetic construct
  VARIANT 1: note = where X at position 1 is a hydroxylated aryl group
  MOD_RES 4: note = X at position 4 is aspartic acid or aspartic acid having its sidechain modified with an O-methyl group
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XFFXFFX

SEQ ID NO: 127 - protein, 7 aa, synthetic construct
  VARIANT 1: note = where X at position 1 is a hydroxylated aryl group
  MOD_RES 5: note = X at position 5 is aspartic acid or aspartic acid having its sidechain modified with an O-methyl group
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XFFFXFX

SEQ ID NO: 128 - skipped

SEQ ID NO: 129 - protein, 7 aa, synthetic construct
  VARIANT 1: note = where X at position 1 is a hydroxylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XXFFFFX

SEQ ID NO: 130 - protein, 7 aa, synthetic construct
  VARIANT 1: note = where X at position 1 is a hydroxylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XXFFKFX

SEQ ID NO: 131 - protein, 7 aa, synthetic construct
  VARIANT 1: note = where X at position 1 is a hydroxylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XXFFKYX

SEQ ID NO: 132 - protein, 7 aa, synthetic construct
  VARIANT 1: note = where X at position 1 is a hydroxylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XXFFYKX

SEQ ID NO: 133 - protein, 7 aa, synthetic construct
  VARIANT 1: note = where X at position 1 is a hydroxylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  VARIANT 7: note = X at position 7 is optional and can be an O-methyl group, a fluorophore, a chemotherapeutic agent, an antiangiogenic agent, a thermoablative nanoparticle, an immunomodulating agent, or an antigen
XXFFFKX

SEQ ID NO: 134 - protein, 8 aa, synthetic construct
  VARIANT 1: note = where X at position 1 is a hydroxylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  VARIANT 8: note = X at position 8 is optional and can be an O-methyl group, a fluorophore, a chemotherapeutic agent, an antiangiogenic agent, a thermoablative nanoparticle, an immunomodulating agent, or an antigen
XXFFFFFX

SEQ ID NO: 135 - protein, 8 aa, synthetic construct
  VARIANT 1: note = where X at position 1 is a hydroxylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  VARIANT 8: note = X at position 8 is optional and can be an O-methyl group, a fluorophore, a chemotherapeutic agent, an antiangiogenic agent, a thermoablative nanoparticle, an immunomodulating agent, or an antigen
XXFFGFFX

SEQ ID NO: 136 - protein, 8 aa, synthetic construct
  VARIANT 1: note = where X at position 1 is a hydroxylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  VARIANT 8: note = X at position 8 is optional and can be an O-methyl group, a fluorophore, a chemotherapeutic agent, an antiangiogenic agent, a thermoablative nanoparticle, an immunomodulating agent, or an antigen
XXFFFGFX

SEQ ID NO: 137 - protein, 8 aa, synthetic construct
  VARIANT 1: note = where X at position 1 is a hydroxylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  VARIANT 8: note = X at position 8 is optional and can be an O-methyl group, a fluorophore, a chemotherapeutic agent, an antiangiogenic agent, a thermoablative nanoparticle, an immunomodulating agent, or an antigen
XXFFFFGX

SEQ ID NO: 138-145 - skipped (8 entries)

SEQ ID NO: 146 - protein, 8 aa, synthetic construct
  VARIANT 1: note = where X at position 1 is a hydroxylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  MOD_RES 7: note = X at position 7 is glutamic acid or glutamic acid having its sidechain modified with an O-methyl group
  VARIANT 8: note = X at position 8 is optional and can be an O-methyl group, a fluorophore, a chemotherapeutic agent, an antiangiogenic agent, a thermoablative nanoparticle, an immunomodulating agent, or an antigen
XXFFFFXX

SEQ ID NO: 147 - protein, 8 aa, synthetic construct
  VARIANT 1: note = where X at position 1 is a hydroxylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  MOD_RES 5: note = X at position 5 is glutamic acid or glutamic acid having its sidechain modified with an O-methyl group
  VARIANT 8: note = X at position 8 is optional and can be an O-methyl group, a fluorophore, a chemotherapeutic agent, an antiangiogenic agent, a thermoablative nanoparticle, an immunomodulating agent, or an antigen
XXFFXFFX

SEQ ID NO: 148 - protein, 8 aa, synthetic construct
  VARIANT 1: note = where X at position 1 is a hydroxylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  MOD_RES 6: note = X at position 6 is glutamic acid or glutamic acid having its sidechain modified with an O-methyl group
  VARIANT 8: note = X at position 8 is optional and can be an O-methyl group, a fluorophore, a chemotherapeutic agent, an antiangiogenic agent, a thermoablative nanoparticle, an immunomodulating agent, or an antigen
XXFFFXFX

SEQ ID NO: 149-152 - skipped (4 entries)

SEQ ID NO: 153 - protein, 8 aa, synthetic construct
  VARIANT 1: note = where X at position 1 is a hydroxylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  MOD_RES 7: note = X at position 7 is aspartic acid or aspartic acid having its sidechain modified with an O-methyl group
  VARIANT 8: note = X at position 8 is optional and can be an O-methyl group, a fluorophore, a chemotherapeutic agent, an antiangiogenic agent, a thermoablative nanoparticle, an immunomodulating agent, or an antigen
XXFFFFXX

SEQ ID NO: 154 - protein, 8 aa, synthetic construct
  VARIANT 1: note = where X at position 1 is a hydroxylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  MOD_RES 5: note = X at position 5 is aspartic acid or aspartic acid having its sidechain modified with an O-methyl group
  VARIANT 8: note = X at position 8 is optional and can be an O-methyl group, a fluorophore, a chemotherapeutic agent, an antiangiogenic agent, a thermoablative nanoparticle, an immunomodulating agent, or an antigen
XXFFXFFX

SEQ ID NO: 155 - protein, 8 aa, synthetic construct
  VARIANT 1: note = where X at position 1 is a hydroxylated aryl group
  VARIANT 2: note = X at position 2 is 3-(1-Naphthyl)-D-alanine, 3-(2-Naphthyl)-D-alanine), (3-(1-Naphthyl)-L-alanine or 3-(2-Naphthyl)-L-alanine
  MOD_RES 6: note = X at position 6 is aspartic acid or aspartic acid having its sidechain modified with an O-methyl group
  VARIANT 8: note = X at position 8 is optional and can be an O-methyl group, a fluorophore, a chemotherapeutic agent, an antiangiogenic agent, a thermoablative nanoparticle, an immunomodulating agent, or an antigen
XXFFFXFX

(19) United States
(12) Patent Application Publication
(10) Pub. No.: US 2025/0206776 A1
(43) Pub. Date: Jun. 26, 2025

(54) ENZYME-INSTRUCTED SELF-ASSEMBLY OF PEPTIDES CONTAINING N-TERMINAL PHOSPHO-AROMATIC CAPPING MOTIF, AND USES THEREOF

(71) Applicant: BRANDEIS UNIVERSITY, Waltham, MA (US)

(72) Inventors: Meihui YI, Waltham, MA (US); Bing XU, Newton, MA (US)

(21) Appl. No.: 18/689,798

(22) PCT Filed: Sep. 9, 2022
(86) PCT No.: PCT/US22/43070

§ 371 Date: Mar. 6, 2024

Related U.S. Application Data
(60) Provisional application No. 63/242,088, filed on Sep. 9, 2021.

(51) Int. Cl. C07K 5/08; A61K 38/00

(52) U.S. Cl. CPC C07K 5/08; A61K 38/00

(57) ABSTRACT

The invention relates to a peptide comprising from 3 to 20 amino acids, including at least two aromatic amino acid residues, and an N-terminal phosphorylated aryl group, wherein upon exposure to an enzyme that hydrolyzes the phosphate group the peptide self-assembles to form nanofibrils and optionally nanoparticles. Also disclosed are self-assembled products formed following exposure of the phospho-aryl peptide to an enzyme that hydrolyzes the phosphate group, and pharmaceutical compositions that contain the phospho-aryl peptide. Methods of using the phospho-aryl peptide include a method for forming a nanofibril network on or near the surface of target cells, a method for collecting a target cell secretome, and a method for treating a cancerous condition.

This application claims the priority benefit of U.S. Provisional Patent Application Ser. No. 63/242,088, filed Sep. 9, 2021, which is hereby incorporated by reference in its entirety.

This invention was made with government support under grant numbers R01 CA142746 and R21 CA252364 awarded by National Institutes of Health (NIH) and under grant numbers DMR-2011846 awarded by National Science Foundation (NSF). The government has certain rights in the invention.

### FIELD OF THE INVENTION

The present invention relates to peptides containing an N-terminal phospho-aromatic capping motif, their use for enzyme-instructed self-assembly to form a nanofibril network on or near the surface of target cells, collecting a target cell secretome, and treating a cancerous condition.

### BACKGROUND OF THE INVENTION

Enzyme-instructed self-assembly (EISA) or enzymatic noncovalent synthesis (He et al., Chem Rev. 120:9994 (2020)), as a versatile approach for mimicking the regulation of noncovalent interactions of biomolecules in a living cell, has emerged as a useful bottom-up strategy for controlling functional supramolecular peptide assemblies (Kim et al., Bioconjugate Chem. 31:492 (2020)), which promise a wide range of potential applications of soft materials in biomedicine, such as tissue engineering (Cui et al., Pept Sci. 94:1 (2010); Shang et al., Chem Commun. 55:5123 (2019)), molecular imaging (Hai et al., Advanced Biosystems 2:1800108 (2018); He et al., Chem Commun. 56:13323 (2020); Yan et al., J Am Chem Soc. 141:10331 (2019)), drug delivery (Mei et al., Chem Commun. 55:4411 (2019); Shi et al., Chem Commun. 51:15265 (2015); Wang et al., Adv Drug Deliver Rev. 110-111:112 (2017)), multimolecular crowding in biosystems (Feng et al., J Am Chem Soc. 140:16433 (2018)), and cancer therapy (Feng et al., Chem, 5:2442 (2019); Jeena et al., Chem Commun. 56:6265 (2020); Tanaka et al., J Am Chem Soc. 137:770 (2015); Yao et al., Nature Communications 9:5032 (2018); Yang et al., Advanced Materials 19:3152 (2007)). Because enzymatic reactions provide a fast and specific transformation of supramolecular peptide assemblies, EISA is particularly attractive for generating non-diffusive supramolecular assemblies (Hendricks et al., Accounts Chem Res. 50:2440 (2017); Ivnitski et al., Angew Chem Int Ed 55:9988 (2016); Ouyang et al., Materials Chemistry Frontiers 4:155 (2020)) in cellular environment (Feng et al., Cell Rep Phys Sci 1:100085 (2020)) for modulating cellular activities (Zhan et al., Chem Commun. 56:6957 (2020)), such as apoptosis (Du et al., Cell Death & Disease 8:e2614 (2017)), morphogenesis (Zhou, X. Du, X. Chen and B. Xu, Biochemistry 57:4867 (2018)), and protein trafficking (Yang et al., Bioconjugate Chem 32(3):502 (2021)).

Most of these studies related to short peptides that utilize phosphotyrosine (ₚTyr) as an enzymatic trigger (Yang et al., Adv Mater 16:1440 (2004)) activated by alkaline phosphatase (ALP) for initiating self-assembly of the peptides, because ALP plays important roles in cell biology and is overexpressed in certain tumours (Fishman et al., Nature 219:697 (1968); Lange et al., Cancer Res. 42:3244 (1982)). Particularly, most of these peptides contain an aromatic capping motif, such as naphthyl (Shi et al., Biomacromolecules 15:3559 (2014)), fluorenyl (Gao et al., J Am Chem Soc. 131:11286 (2009)), or pyrenyl (Li et al., Angew. Chem. Int. Ed. 57:11716 (2018)) group at the N-terminal, and L- or D-pTyr at the C-terminal or in the middle of the peptides (Li et al., Angew. Chem. Int. Ed. 57:11716 (2018); Wang et al., Soft Matter 7:3897 (2011); Yao et al., ACS Nano 14:4882 (2020); Zheng et al., J Am Chem Soc. 138: 11128 (2016)). A representative example of these EISA substrates is Nap-ffₚy (1P) (see FIG. 1A), which carries a D-ₚTyr (ₚy) at the C-terminal of the peptide. 1P has revealed many key features of EISA and led to unexpected formation of pericellular nanofibers that selectively kill cancer cells (Kuang et al., Angew Chem Int Ed Engl. 53:8104 (2014)). On the other hand, the phosphate trigger at N-terminal of peptide is much less explored, except the work of Ye et al. that employs a phosphorylated dye at the N-terminal of a peptide (Yan et al., J Am Chem Soc. 141:10331 (2019)), but the rate of dephosphorylation is relatively slow.

It would be desirable, therefore, to identify functionalized peptide structures having a novel modified N-terminal capping motif, whereby the peptides exhibit improved self-assembly characteristics to facilitate both in vivo and ex vivo uses thereof.

### SUMMARY OF THE INVENTION

A first aspect of the invention relates to a peptide including from 3 to 20 amino acids, including at least two aromatic amino acid residues, and an N-terminal phosphorylated aryl group, wherein upon exposure to an enzyme that hydrolyzes the phosphate group the peptide self-assembles to form nanofibrils and optionally nanoparticles.

A second aspect of the invention relates to a product formed by exposing a peptide according to the first aspect to an enzyme that hydrolyzes the phosphate group. Upon hydrolysis of the phosphate group, the resulting N-terminal aryl group promotes self-assembly of the peptide to form larger structures including, without limitation, nanoparticles, nanofibers, amorphous sheets, and hydrogel networks.

A third aspect of the invention relates to a pharmaceutical composition including a pharmaceutically acceptable carrier and a peptide according to the first aspect of the invention.

A fourth aspect of the invention relates to a method for forming a nanofibril network on or near the surface of target cells. This method includes the steps of: contacting a target cell that expresses a cell surface-bound enzyme having hydrolytic activity, secretes an enzyme having hydrolytic activity, or both, with the peptide according to the first aspect of the invention or the pharmaceutical composition according to the third aspect of the invention, wherein said contacting is effective to hydrolyze the phosphate group and cause in situ self-assembly of the peptides to form a nanofibril network on or near the surface of the target cell.

A fifth aspect of the invention relates to a method for collecting a target cell secretome including the steps of: contacting a target cell that expresses a cell surface-bound enzyme having hydrolytic activity, secretes an enzyme having hydrolytic activity, or both, with a peptide according to the first aspect of the invention or a pharmaceutical composition according to the third aspect of the invention, wherein said contacting is effective to hydrolyze the phosphate group and cause in situ self-assembly of the peptide to form a nanofibril network on or near the surface of the target cell, whereby the nanofibril network retains the target cell secretome from the pericellular space of the target cell; separating the target cell secretome from the nanofibril network; and collecting the separated target cell secretome.

A sixth aspect of the invention relates to a method for treating a cancerous condition including the steps of: administering to a subject having a cancerous condition a therapeutically effective amount of a peptide according to the first aspect of the invention or a pharmaceutical composition according to the third aspect of the invention, wherein said administering is effective to hydrolyze the phosphate group and cause in vivo self-assembly of the peptides to form a nanofibril network on or near the surface of cancer cells. The formation of the nanofibril network on or near the surface of cancer cells can disrupt one or more of cancer cell motility, cancer cell signaling, and cancer cell survival.

The accompanying Examples demonstrate the successful use of N-terminal phosphorylated aromatic moieties to control self-assembly of peptides. While the three ff derivatives (2, 4, 6) are unable to form hydrogels, the three fff derivatives (3, 5, 7) result in hydrogels after ALP-catalysed dephosphorylation converting the nanoparticles made of the precursors to the nanofibers consisted of the corresponding hydrogelators (i.e., 3, 5, or 7). Rheological evaluation shows that the resulting three hydrogels have relatively high storage moduli, up to 10⁴ Pa, when the concentrations of the hydrogelators are about 8 mM (about 0.5 wt %). Moreover, phosphobiphenyl carboxylic acid (ₚBP) and phosphonaphthoic acid (ₚNP) act as faster enzyme triggers than phospho-_{D}Tyr (ₚy) and phosphohydroxybenzoic acid (ₚB) for hydrogelation. As the first example to show the dephosphorylation of ₚBP and ₚNP for rapid enzymatic self-assembly and hydrogelation, this work offers a novel molecular platform and identifies fast triggers for EISA catalyzed by ALP.

### BRIEF DESCRIPTION OF THE DRAWINGS

FIG. 1A illustrates the structure of a prior peptide derivative bearing a phosphate at the C-terminal end, designated 1P (see Kuang et al., Angew Chem Int Ed Engl. 53:8104 (2014), which is hereby incorporated by reference in its entirety). FIG. 1B illustrates the structures of peptide derivatives bearing a phosphate at the N-terminal end, designated 2P-7P.

FIG. 2 illustrates a synthesis scheme for 2P. The same procedures were used to synthesize 3P-7P.

FIG. 3 illustrates a panel of optical images of 2P, 3P, 4P, 5P, 6P and 7P (0.5 wt % in PBS buffer, pH7.4) before and after incubation with ALP (1 UmL⁻¹) for 24 h.

FIG. 4 illustrates a panel of dynamic time sweeps of 3P, 5P, and 7P, all at 8 mM, incubated with ALP at 1 and 0.1 UmL⁻¹ and at a strain of 1% and frequency of 6.28 rads⁻¹.

FIG. 5A is a panel of frequency sweeps of 3P, 5P, and 7P, all at 8 mM, conducted after 24 h incubation with ALP at 1.0 and 0.1 UmL⁻¹ and at the strain of 1%. FIG. 5B is a panel of dynamic strain sweeps of 3P, 5P, and 7P, all at 8 mM, conducted after 24 h incubation with ALP at 1.0 and 0.1 UmL⁻¹ and at the frequency of 6.28 rads⁻¹.

FIG. 6 is a panel of TEM images of 2P, 3P, 4P, 5P, 6P and 7P at 8 mM before and after ALP treatment. The concentration of ALP is 0.1 UmL⁻¹. The duration time is 24 h. The scale bar is 100 nm.

FIG. 7 is a pair of SEM images of 3P and 7P at 8 mM after ALP treatment for over one week. The concentration of ALP was 0.1 UmL⁻¹.

FIG. 8 illustrates the molecular structures of 8P-11P, with 8P bearing a naphthyl N-terminal capping group and phosphotyrosine (like 1P in FIG. 1A) and 9P-11P bearing a pBP N-terminal capping group.

FIG. 9 is a graph depicting the IC₅₀ values of 9P and 10P incubated with Saos2 and SJSA1 cells.

FIG. 10 is a graph depicting the IC₅₀ values of 11P incubated with Saos2 and SJSA1 cells.

FIG. 11 is a panel of graphs illustrating the cell viability of Saos2 and SJSA1 treated with 9P, 10P of different concentration without and with the coincubation of DQB. The concentration of DQB is 20 μM. The duration time is 2 h.

FIG. 12A illustrates the molecular structures of 9 and 10, the dephosphorylated variant of 9P and 10P, respectively. FIG. 12B is a graph illustrating the cell viability of Saos2 and SJSA1 treated with 9 and 10. FIG. 12C is a graph illustrating the IC₅₀ values of 9 and 10 incubated with Saos2 and SJSA1.

### DETAILED DESCRIPTION OF THE INVENTION

One aspect of the invention relates to a peptide, preferably comprising from 3 to 20 amino acids, including at least two aromatic amino acid residues and an N-terminal phosphorylated aryl group, wherein upon exposure to an enzyme that hydrolyzes the phosphate group the peptide self-assembles to form nanofibrils and optionally nanoparticles.

The peptide can have any length as long as the conjugate is capable of self-assembly. In certain embodiments, the peptide preferably contains from 3 up to about 20 amino acids, including from 3 to 15 amino acids, from 3 to 12 amino acids, from 3 to 10 amino acids, or from 3 to 8 amino acids. Thus, peptides that contain 3, 4, 5, 6, 7, 8, 9, 10, 11, 12, 13, 14, 15, 16, 17, 18, 19, or 20 amino acids are contemplated.

The peptides can include all D-amino acids, all L-amino acids, or a mixture of L-amino acids and D-amino acids. In preferred embodiments, the peptide includes only D-amino acids or a mixture of D-amino acids and L-amino acids where the D-amino acid content is greater than 50%, 60%, 70%, 80%, 90%, or 95%.

The amino acid residues that form the peptide can be any naturally occurring or non-naturally occurring amino acid, but preferably the peptide includes two or more aromatic amino acids as described above. Any natural or non-natural aromatic amino acids can be present. Exemplary aromatic amino acids include any one or more of tyrosine, phenylalanine, L-3,4-dihydroxyphenylalanine, napthylalanine, tryptophan, 5-hydroxytryptophan, and histidine as well as any other phenylalanine derivatives, napthylalanine derivatives, tyrosine derivatives, and tryptophan derivatives. In certain embodiments, at least 40%, at least 50%, at least 60%, at least 70%, at least 80%, at least 90%, or 100% of the amino acid residues in the peptide are aromatic amino acid residues.

In certain embodiments, the peptide can include a fluorophore, a cytotoxic agent such as a chemotherapeutic agent, an antiangiogenic agent, or an immunomodulating agent, an antibiotic, an antigen, or a thermoablative (paramagnetic) particle coupled to a C-terminus of the peptide.

In certain embodiments, the peptide can include one or more amino acids whose sidechain is easily conjugated to, e.g., a fluorophore, a cytotoxic agent such as a chemotherapeutic agent, an antiangiogenic agent, or an immunomodulating agent, an antibiotic, an antigen, or a thermoablative (paramagnetic) particle. Numerous examples of each of these categories are well known in the art.

Exemplary amino acids that can be derivatized include lysine or arginine, whose terminal amino group of its side chain is reactive in conjugation procedures. Examples of conjugating a chemotherapeutic agent (e.g., doxorubicin, daunorubicin, taxol) to a Lys sidechain are described in DeFeo-Jones et al., Nature Med. 6(11):1248-52 (2000), Schreier et al., PlosOne 9(4):e94041 (2014), Gao et al., J Am Chem Soc. 131:13576 (2009), each of which is hereby incorporated by reference in its entirety. An examples of conjugating a fluorophore, such as 4-nitro-2,1,3-benzoxadiazole (“NBD”) to a Lys sidechain is described in Gao et al., Nat. Commun 3:1033 (2012), which is hereby incorporated by reference in its entirety.

In general, guanidine groups present in arginine can be reacted with reagents possessing guanidine-reactive groups using known reaction schemes. Exemplary guanidine reactive functional groups include, without limitation, NHS esters using gas phase synthesis (McGee et al., J. Am. Chem. Soc., 134 (28):11412-11414 (2012), which is hereby incorporated by reference in its entirety).

In general, thiol groups present in cysteine (or cysteine derivative) side chains can be reacted with reagents possessing thiol-reactive functional groups using known reaction schemes. Exemplary thiol-reactive functional groups include, without limitation, iodoacetamides, maleimides, and alkyl halides. Reagents to be conjugated include those listed above.

In general, carboxyl groups present in glutamic or aspartic acid side chains, or at the C-terminal amino acid residue, can be reacted with reagents possessing carboxyl-reactive functional groups using known reaction schemes. Exemplary carboxyl-reactive functional groups include, without limitation, amino groups, amines, bifunctional amino linkers. Reagents to be conjugated include those listed above.

Exemplary peptide sequences include, without limitation, fff or FFF, ffff or FFFF (SEQ ID NO:1), ffkf or FFKF (SEQ ID NO:2), ffky or FFKY (SEQ ID NO:3), ffyk or FFYK (SEQ ID NO:4), fffk or FFFK (SEQ ID NO:5), fffff or FFFFF (SEQ ID NO:6), ffgff or FFGFF (SEQ ID NO:7), fffgf or FFFGF (SEQ ID NO:8), ffffg or FFFFG (SEQ ID NO:9), ffe or FFE, fffe or FFFE (SEQ ID NO:10), ffke or FFKE (SEQ ID NO:11), ffek or FFEK (SEQ ID NO:12), ffffe or FFFFE (SEQ ID NO:13), ffeff or FFEFF (SEQ ID NO:14), fffef or FFFEF (SEQ ID NO:15), ffd or FFD, fffd or FFFD (SEQ ID NO:16), ffkd or FFKD (SEQ ID NO:17), ffdk or FFDK (SEQ ID NO:18), ffffd or FFFFD (SEQ ID NO:19), ffdff or FFDFF (SEQ ID NO:20), fffdf or FFFDF (SEQ ID NO:21), nal-fff or Nal-FFF (SEQ ID NO:22), nal-ffff or Nal-FFFF (SEQ ID NO:23), nal-ffkf or Nal-FFKF (SEQ ID NO:24), nal-ffky or Nal-FFKY (SEQ ID NO:25), nal-ffyk or Nal-FFYK (SEQ ID NO:26), nal-fffk or Nal-FFFK (SEQ ID NO:27), nal-fffff or Nal-FFFFF (SEQ ID NO:28), nal-ffgff or Nal-FFGFF (SEQ ID NO:29), nal-fffgf or Nal-FFFGF (SEQ ID NO:30), nal-ffffg or Nal-FFFFG (SEQ ID NO:31), nal-ffe or Nal-FFE (SEQ ID NO:32), nal-fffe or Nal-FFFE (SEQ ID NO:33), nal-ffke or Nal-FFKE (SEQ ID NO:34), nal-ffek or Nal-FFEK (SEQ ID NO:35), nal-ffffe or Nal-FFFFE (SEQ ID NO:36), nal-ffeff or Nal-FFEFF (SEQ ID NO:37), nal-fffef or Nal-FFFEF (SEQ ID NO:38), nal-ffd or Nal-FFD (SEQ ID NO:39), nal-fffd or Nal-FFFD (SEQ ID NO:40), nal-ffkd or Nal-FFKD (SEQ ID NO:41), nal-ffdk or Nal-FFDK (SEQ ID NO:42), nal-ffffd or Nal-FFFFD (SEQ ID NO:43), nal-ffdff or Nal-FFDFF (SEQ ID NO:44), and nal-fffdf or Nal-FFFDF (SEQ ID NO:45), where lowercase letters denote D-amino acids, uppercase letters denote L-amino acids, nal is a D-naphthylalanine (3-(1-Naphthyl)-D-alanine or 3-(2-Naphthyl)-D-alanine), and Nal is an L-naphthylalanine (3-(1-Naphthyl)-L-alanine or 3-(2-Naphthyl)-L-alanine). Acidic residues such as D- or L-aspartic acid and D- or L-glutamic acid can optionally have their sidechains O-methylated. Further, the C-terminal carboxylic acid can optionally be O-methylated.

Any of a variety of N-terminal phosphorylated aryl groups can be used in the peptides of the present invention. Preferably, the N-terminal phosphorylated aryl group is a phosphobisaromatic group or phosphotrisaromatic group, although larger fused or multi-ring aromatic groups can also be used.

Exemplary phosphobisaromatic groups include, without limitation:

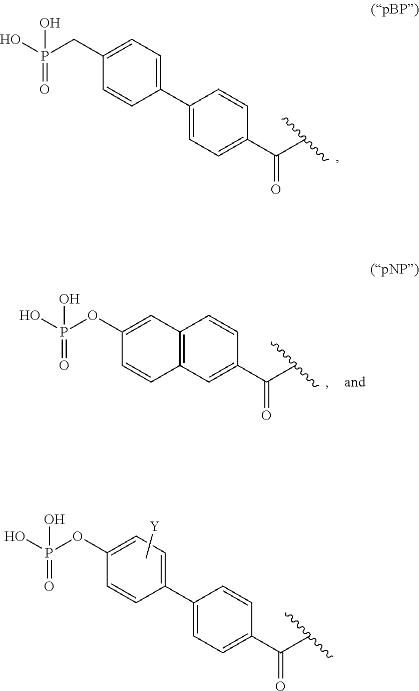

where Y is —F, —Cl, —Br, or —CN.

Exemplary phosphotrisaromatic groups include, without limitation:

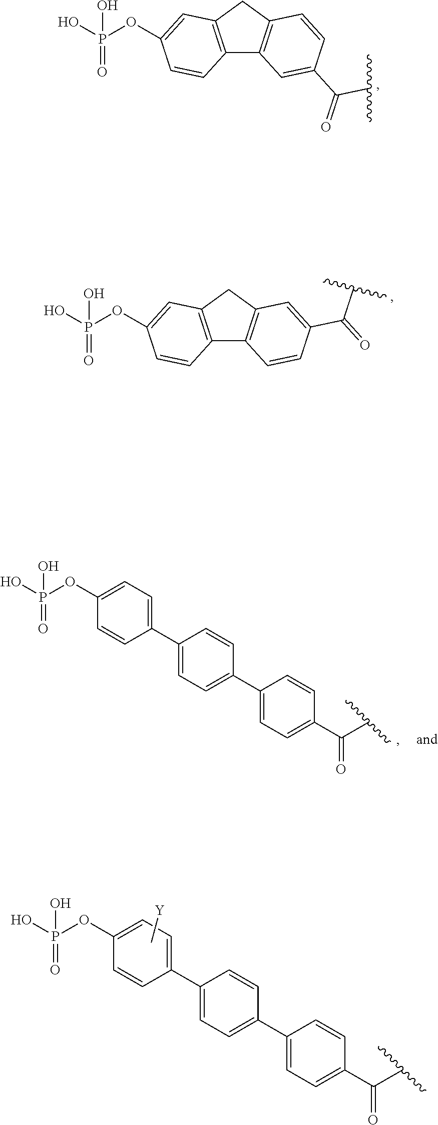

where Y is —F, —Cl, —Br, or —CN.

These phosphobisaromatic groups and phosphotrisaromatic groups result from the reaction of the carboxylic acid intermediate with the N-terminal end of the peptide (forming a peptide bond). The carboxylic acid intermediates can be prepared by converting the corresponding hydroxyl-bearing aromatic carboxylic acid to the phospho intermediate using the previously reported procedures of Graber et al., ACS Chemical Biology 6:1008 (2011), which is hereby incorporated by reference in its entirety. The hydroxy-triphenyl carboxylic acid can be prepared by reacting 4′-bromo-[1,1′-biphenyl]-4-ol (1 eq) and 4-boronobenzoic acid (1.2 eq), Na₂CO₃ (3 eq.), in solvent (Methanol:water=4:1) with Pd/C catalyst (0.1 mol %) for 24 hr using a magnetic stirrer, reflux condenser, and 85° C. oil bath. The 7-hydroxy-fluorene-2-carboxylic acid and 7-hydroxy-fluorene-3-carboxylic acid can be prepared using the previously reported procedures by Ishikawa et al., Nippon Kagaku Zasshi 81:1289-92 (1960), which is hereby incorporated by reference in its entirety. The same phosphorylating procedures in the Examples can be used to phosphorylate the 9-hydroxy-fluorene-3-carboxylic acid or 9-hydroxy-fluorene-2-carboxylic acid.

Exemplary phospho-aryl peptides include, without limitation: X₁-fff-Z₁ or X₁-FFF-Z₁, X₁-ffff-Z₁ or X₁-FFFF-Z₁ (SEQ ID NO:46), X₁-ffkf-Z₁ or X₁-FFKF-Z₁ (SEQ ID NO:47), X₁-ffky-Z₁ or X₁-FFKY-Z₁ (SEQ ID NO:48), X₁-ffyk-Z₁ or X₁-FFYK-Z₁ (SEQ ID NO:49), X₁-fffk-Z₁ or X₁-FFFK-Z₁ (SEQ ID NO:50), X₁-fffff-Z₁ or X₁—FFFFF-Z₁ (SEQ ID NO:51), X₁-ffgff-Z₁ or X₁-FFGFF-Z₁ (SEQ ID NO:52), X₁-fffgf-Z₁ or X₁-FFFGF-Z₁ (SEQ ID NO:53), X₁-ffffg-Z₁ or X₁-FFFFG-Z₁(SEQ ID NO:54), X₁-ffk(Z₂)f or X₁-FFK(Z₂)F (SEQ ID NO:55), X₁-ffk(Z₂)y or X₁-FFK(Z₂)Y (SEQ ID NO:56), X₁-ffyk(Z₂) or X₁-FFYK(Z₂) (SEQ ID NO:57), X₁-fffk(Z₂) or X₁-FFFK(Z₂) (SEQ ID NO:58), X₁-ffe(Z₃)-Z₁ or X₁-FFE(Z₃)-Z₁ (SEQ ID NO:59), X₁-fffe(Z₃)-Z₁ or X₁-FFFE(Z₃)-Z₁ (SEQ ID NO:60), X₁-ffk(Z₂)e(Z₃)-Z₁ or X₁-FFK(Z₂)E(Z₃)-Z₁ (SEQ ID NO:61), X₁-ffe(Z₃)k(Z₂)-Z₁ or X₁-FFE(Z₃)K(Z₂)-Z₁ (SEQ ID NO:62), X₁-ffffe(Z₃)-Z₁ or X₁-FFFFE(Z₃)-Z₁ (SEQ ID NO:63), X₁-ffe(Z₃)ff-Z₁ or X₁-FFE(Z₃)FF-Z₁ (SEQ ID NO:64), X₁-fffe(Z₃)f-Z₁ or X₁-FFFE(Z₃)F-Z₁ (SEQ ID NO:65), X₁-ffd(Z₃)-Z₁ or X₁-FFD(Z₃)-Z₁ (SEQ ID NO:66), X₁-fffd(Z₃)-Z₁ or X₁-FFFD(Z₃)-Z₁ (SEQ ID NO:67), X₁-ffk(Z₂)d(Z₃)-Z₁ or X₁-FFK(Z₂)D(Z₃)-Z₁(SEQ ID NO:68), X₁-ffd(Z₃)k(Z₂)-Z₁ or X₁-FFD(Z₃)K(Z₂)-Z₁ (SEQ ID NO:69), X₁-ffffd(Z₃)-Z₁ or X₁-FFFFD(Z₃)-Z₁ (SEQ ID NO:70), X₁-ffd(Z₃)ff-Z₁ or X₁-FFD(Z₃)FF-Z₁ (SEQ ID NO:71), X₁-fffd(Z₃)f-Z₁ or X₁-FFFD(Z₃)F-Z₁ (SEQ ID NO:72), X₁-nal-fff-Z₁ or X₁-Nal-FFF-Z₁ (SEQ ID NO:73), X₁-nal-ffff-Z₁ or X₁-Nal-FFFF-Z₁ (SEQ ID NO:74), X₁-nal-ffkf-Z₁ or X₁-Nal-FFKF-Z₁ (SEQ ID NO:75), X₁-nal-ffky-Z₁ or X₁-Nal-FFKY-Z₁ (SEQ ID NO:76), X₁-nal-ffyk-Z₁ or X₁-Nal-FFYK-Z₁ (SEQ ID NO:77), X₁-nal-fffk-Z₁ or X₁-Nal-FFFK-Z₁ (SEQ ID NO:78), X₁-nal-fffff-Z₁ or X₁-Nal-FFFFF-Z₁ (SEQ ID NO:79), X₁-nal-ffgff-Z₁ or X₁-Nal-FFGFF-Z₁ (SEQ ID NO:80), X₁-nal-fffgf-Z₁ or X₁-Nal-FFFGF-Z₁ (SEQ ID NO:81), X₁-nal-ffffg-Z₁ or X₁-Nal-FFFFG-Z₁ (SEQ ID NO:82), X₁-nal-ffk(Z₂)f or X₁-Nal-FFK(Z₂)F (SEQ ID NO:83), X₁-nal-ffk(Z₂)y or X₁-Nal-FFK(Z₂)Y (SEQ ID NO:84), X₁-nal-ffyk(Z₂) or X₁-Nal-FFYK(Z₂) (SEQ ID NO:85), X₁-nal-fffk(Z₂) or X₁-Nal-FFFK(Z₂) (SEQ ID NO:86), X₁-nal-ffe(Z₃)-Z₁ or X₁-Nal-FFE(Z₃)-Z₁ (SEQ ID NO:87), X₁-nal-fffe(Z₃)-Z₁ or X₁-Nal-FFFE(Z₃)-Z₁ (SEQ ID NO:88), X₁-nal-ffk(Z₂)e(Z₃)-Z₁ or X₁-Nal-FFK(Z₂)E(Z₃)-Z₁ (SEQ ID NO:89), X₁-nal-ffe(Z₃)k(Z₂)-Z₁ or X₁-Nal-FFE(Z₃)K(Z₂)-Z₁ (SEQ ID NO:90), X₁-nal-ffffe(Z₃)-Z₁ or X₁-Nal-FFFFE(Z₃)-Z₁ (SEQ ID NO:91), X₁-nal-ffe(Z₃)ff-Z₁ or X₁-Nal-FFE(Z₃)FF-Z₁ (SEQ ID NO:92), X₁-nal-fffe(Z₃)f-Z₁ or X₁-Nal-FFFE(Z₃)F-Z₁ (SEQ ID NO:93), X₁-nal-ffd(Z₃)-Z₁ or X₁-Nal-FFD(Z₃)-Z₁ (SEQ ID NO:94), X₁-nal-fffd(Z₃)-Z₁ or X₁-Nal-FFFD(Z₃)-Z₁ (SEQ ID NO:95), X₁-nal-ffk(Z₂)d(Z₃)-Z₁ or X₁-Nal-FFK(Z₂)D(Z₃)-Z₁ (SEQ ID NO:96), X₁-nal-ffd(Z₃)k(Z₂)-Z₁ or X₁-Nal-FFD(Z₃)K(Z₂)-Z₁ (SEQ ID NO:97), X₁-nal-ffffd(Z₃)-Z₁ or X₁-Nal-FFFFD(Z₃)-Z₁ (SEQ ID NO:98), X₁-nal-ffd(Z₃)ff-Z₁ or X₁-Nal-FFD(Z₃)FF-Z₁ (SEQ ID NO:99), and X₁-nal-fffd(Z₃)f-Z₁ or X₁-Nal-FFFD(Z₃)F-Z₁ (SEQ ID NO:100), wherein lowercase letters denote D-amino acids, uppercase letters denote L-amino acids, nal is a D-naphthylalanine (3-(1-Naphthyl)-D-alanine or 3-(2-Naphthyl)-D-alanine), and Nal is an L-naphthylalanine (3-(1-Naphthyl)-L-alanine or 3-(2-Naphthyl)-L-alanine);
X₁ is

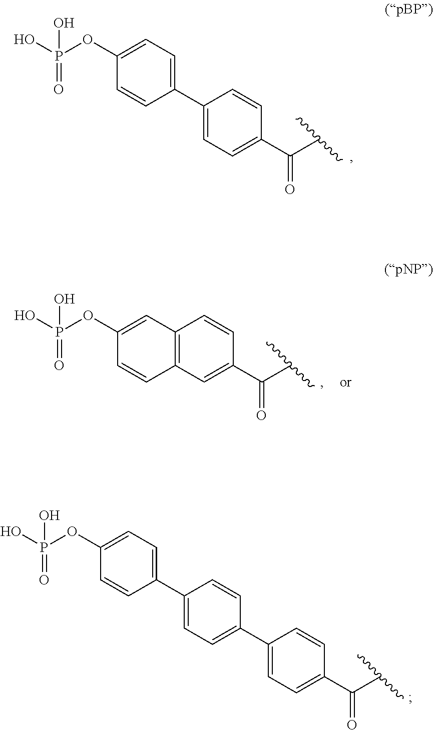 [SMILES: *C(=O)c1ccc(-c2ccc(OP(=O)(O)O)cc2)cc1 ; *C(=O)c1ccc2cc(OP(=O)(O)O)ccc2c1 ; *C(=O)c1ccc(-c2ccc(-c3ccc(OP(=O)(O)O)cc3)cc2)cc1]

Z₁ is selected from the group of —OH (the unmodified C-terminal amino acid residue), —O—CH₃ (methylation of the C-terminal amino acid residue), a fluorophore, a chemotherapeutic agent, an antiangiogenic agent, a thermoablative nanoparticle, an immunomodulating agent, or an antigen;
Z₂ is optional and is selected from the group of —H (the unmodified amino group on the lysine sidechain), a fluorophore, a chemotherapeutic agent, an antiangiogenic agent, a thermoablative nanoparticle, an immunomodulating agent, or an antigen; and
Z₃ is selected from the group of —OH (the unmodified acidic side chain) and —O—CH₃ (methylation of the acidic side chain).

The phospho-aryl peptides of the present invention can be synthesized using standard peptide synthesis operations. These include both 9-Fluorenylmethyloxy-carbonyl (“FMOC”) and tert-Butyl oxy carbonyl (“tBoc”) synthesis protocols that can be carried out on automated solid phase peptide synthesis instruments including, without limitation, the Applied Biosystems 431A, 433A synthesizers and Peptide Technologies Symphony or large scale Sonata or CEM Liberty automated solid phase peptide synthesizers. This can be followed with standard HPLC purification to achieve a purified peptide product.

A related aspect of the invention relates to the product formed by exposing the phospho-aryl peptide of the invention to an enzyme that hydrolyzes the phosphate group.

Exemplary dephosphorylated aryl peptides include, without limitation: X₂-fff-Z₁ or X₂-FFF-Z₁, X₂-ffff-Z₁ or X₂-FFFF-Z₁(SEQ ID NO:101), X₂-ffkf-Z₁ or X₂-FFKF-Z₁ (SEQ ID NO:102), X₂-ffky-Z₁ or X₂-FFKY-Z₁(SEQ ID NO:103), X₂-ffyk-Z₁ or X₂-FFYK-Z₁ (SEQ ID NO:104), X₂-fffk-Z₁ or X₂-FFFK-Z₁(SEQ ID NO:105), X₂-fffff-Z₁ or X₂-FFFFF-Z₁ (SEQ ID NO:106), X₂-ffgff-Z₁ or X₂-FFGFF-Z₁(SEQ ID NO:107), X₂-fffgf-Z₁ or X₂-FFFGF-Z₁ (SEQ ID NO:108), X₂-ffffg-Z₁ or X₂-FFFFG-Z₁ (SEQ ID NO:109), X₂-ffk(Z₂)f or X₂-FFK(Z₂)F (SEQ ID NO:110), X₁-ffk(Z₂)y or X₂-FFK(Z₂)Y (SEQ ID NO:111), X₂-ffyk(Z₂) or X₂-FFYK(Z₂) (SEQ ID NO: 112), X₂-fffk(Z₂) or X₂-FFFK(Z₂) (SEQ ID NO:113), X₂-ffe(Z₃)-Z₁ or X₂-FFE(Z₃)-Z₁ (SEQ ID NO:114), X₂-fffe(Z₃)-Z₁ or X₂-FFFE(Z₃)-Z₁ (SEQ ID NO:115), X₂-ffk(Z₂)e(Z₃)-Z₁ or X₂-FFK(Z₂)E(Z₃)-Z₁ (SEQ ID NO:116), X₂-ffe(Z₃)k(Z₂)-Z₁ or X₂-FFE(Z₃)K(Z₂)-Z₁ (SEQ ID NO:117), X₂-ffffe(Z₃)-Z₁ or X₂-FFFFE(Z₃)-Z₁ (SEQ ID NO:118), X₂-ffe(Z₃)ff-Z₁ or X₂-FFE(Z₃)FF-Z₁ (SEQ ID NO:119), X₂-fffe(Z₃)f-Z₁ or X₂-FFFE(Z₃)F-Z₁ (SEQ ID NO:120), X₂-ffd(Z₃)-Z₁ or X₂-FFD(Z₃)-Z₁ (SEQ ID NO:121), X₂-fffd(Z₃)-Z₁ or X₂-FFFD(Z₃)-Z₁(SEQ ID NO:122), X₂-ffk(Z₂)d(Z₃)-Z₁ or X₂-FFK(Z₂)D(Z₃)-Z₁ (SEQ ID NO:123), X₂-ffd(Z₃)k(Z₂)-Z₁ or X₂-FFD(Z₃)K(Z₂)-Z₁ (SEQ ID NO:124), X₂-ffffd(Z₃)-Z₁ or X₂-FFFFD(Z₃)-Z₁ (SEQ ID NO:125), X₂-ffd(Z₃)ff-Z₁ or X₂-FFD(Z₃)FF-Z₁ (SEQ ID NO:126), X₂-fffd(Z₃)f-Z₁ or X₂-FFFD(Z₃)F-Z₁ (SEQ ID NO:127), X₂-nal-fff-Z₁ or X₂-Nal-FFF-Z₁ (SEQ ID NO:128), X₂-nal-ffff-Z₁ or X₂-Nal-FFFF-Z₁ (SEQ ID NO:129), X₂-nal-ffkf-Z₁ or X₂-Nal-FFKF-Z₁ (SEQ ID NO:130), X₂-nal-ffky-Z₁ or X₂-Nal-FFKY-Z₁ (SEQ ID NO:131), X₂-nal-ffyk-Z₁ or X₂-Nal-FFYK-Z₁ (SEQ ID NO:132), X₂-nal-fffk-Z₁ or X₂-Nal-FFFK-Z₁ (SEQ ID NO:133), X₂-nal-fffff-Z₁ or X₂-Nal-FFFFF-Z₁ (SEQ ID NO:134), X₂-nal-ffgff-Z₁ or X₂-Nal-FFGFF-Z₁ (SEQ ID NO:135), X₂-nal-fffgf-Z₁ or X₂-Nal-FFFGF-Z₁ (SEQ ID NO:136), X₂-nal-ffffg-Z₁ or X₂-Nal-FFFFG-Z₁ (SEQ ID NO:137), X₂-nal-ffk(Z₂)f or X₁-Nal-FFK(Z₂)F (SEQ ID NO:138), X₂-nal-ffk(Z₂)y or X₁-Nal-FFK(Z₂)Y (SEQ ID NO:139), X₂-nal-ffyk(Z₂) or X₁-Nal-FFYK(Z₂) (SEQ ID NO:140), X₂-nal-fffk(Z₂) or X₁-Nal-FFFK(Z₂) (SEQ ID NO:141), X₂-nal-ffe(Z₃)-Z₁ or X₂-Nal-FFE(Z₃)-Z₁ (SEQ ID NO:142), X₂-nal-fffe(Z₃)-Z₁ or X₂-Nal-FFFE(Z₃)-Z₁ (SEQ ID NO:143), X₂-nal-ffk(Z₂)e(Z₃)-Z₁ or X₂-Nal-FFK(Z₂)E(Z₃)-Z₁ (SEQ ID NO:144), X₂-nal-ffe(Z₃)k(Z₂)-Z₁ or X₂-Nal-FFE(Z₃)K(Z₂)-Z₁ (SEQ ID NO:145), X₂-nal-ffffe(Z₃)-Z₁ or X₂-Nal-FFFFE(Z₃)-Z₁ (SEQ ID NO:146), X₂-nal-ffe(Z₃)ff-Z₁ or X₂-Nal-FFE(Z₃)FF-Z₁ (SEQ ID NO:147), X₂-nal-fffe(Z₃)f-Z₁ or X₂-Nal-FFFE(Z₃)F-Z₁ (SEQ ID NO:148), X₂-nal-ffd(Z₃)-Z₁ or X₂-Nal-FFD(Z₃)-Z₁ (SEQ ID NO:149), X₂-nal-fffd(Z₃)-Z₁ or X₂-Nal-FFFD(Z₃)-Z₁ (SEQ ID NO:150), X₂-nal-ffk(Z₂)d(Z₃)-Z₁ or X₂-Nal-FFK(Z₂)D(Z₃)-Z₁ (SEQ ID NO:151), X₂-nal-ffd(Z₃)k(Z₂)-Z₁ or X₂-Nal-FFD(Z₃)K(Z₂)-Z₁ (SEQ ID NO:152), X₂-nal-ffffd(Z₃)-Z₁ or X₂-Nal-FFFFD(Z₃)-Z₁ (SEQ ID NO:153), X₂-nal-ffd(Z₃)ff-Z₁ or X₂-Nal-FFD(Z₃)FF-Z₁ (SEQ ID NO:154), and X₂-nal-fffd(Z₃)f-Z₁ or X₂-Nal-FFFD(Z₃)F-Z₁ (SEQ ID NO:155);
wherein
X₂ is

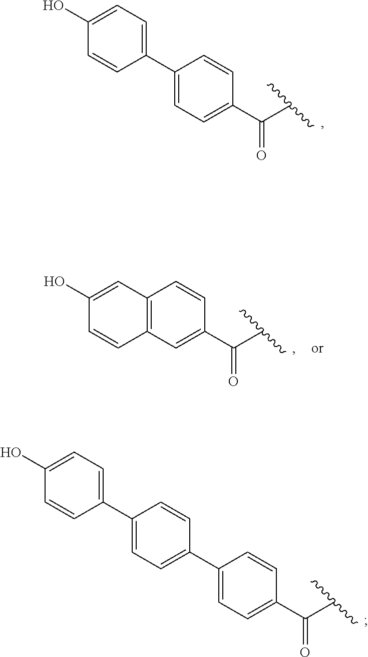 [SMILES: *C(=O)c1ccc(-c2ccc(O)cc2)cc1 ; *C(=O)c1ccc2cc(O)ccc2c1 ; *C(=O)c1ccc(-c2ccc(-c3ccc(O)cc3)cc2)cc1]

Z₁ is selected from the group of —OH (the unmodified C-terminal amino acid residue), —O—CH₃ (methylation of the C-terminal amino acid residue), a fluorophore, a chemotherapeutic agent, an antiangiogenic agent, a thermoablative nanoparticle, an immunomodulating agent, or an antigen;
Z₂ is optional and is selected from the group of —H (the unmodified amino group on the lysine sidechain), a fluorophore, a chemotherapeutic agent, an antiangiogenic agent, a thermoablative nanoparticle, an immunomodulating agent, or an antigen; and
Z₃ is selected from the group of —OH (the unmodified acidic side chain) and —O—CH₃ (methylation of the acidic side chain).

The dephosphorylated aryl peptides are capable of self-assembly and hydrogelation. Thus, one aspect of the invention relates to self-assembled nanoparticles and nanofibers, and supermolecular hydrogels.

A further aspect of the invention relates to a pharmaceutical composition comprising a pharmaceutically acceptable carrier and a phospho-aryl peptide of the invention, which is present in an effective amount.

In certain embodiments, more than one peptide can be provided. The peptides can similar in structure, but possess different conjugated agents as described above. In alternative embodiments, the peptides can be structurally distinct, including different structures that are nevertheless capable of self-assembly due to the structural compatibility of the aromatic amino acids residues in the different peptides.

In certain embodiments, the carrier is an aqueous medium that is well tolerated for administration to an individual, typically a sterile isotonic aqueous buffer. Exemplary aqueous media include, without limitation, normal saline (about 0.9% NaCl), phosphate buffered saline (PBS), sterile water/distilled autoclaved water (DAW), as well as cell growth medium (e.g., MEM, with or without serum), aqueous solutions of dimethyl sulfoxide (DMSO), polyethylene glycol (PEG), and/or dextran (less than 6% per by weight.)

To improve patient tolerance to administration, the pharmaceutical composition preferably has a pH of about 6 to about 8, preferably about 6.5 to about 7.4. Typically, sodium hydroxide and hydrochloric acid are added as necessary to adjust the pH.

The pharmaceutical composition suitably includes a weak acid or salt as a buffering agent to maintain pH. Citric acid has the ability to chelate divalent cations and can thus also prevent oxidation, thereby serving two functions as both a buffering agent and an antioxidant stabilizing agent. Citric acid is typically used in the form of a sodium salt, typically 10-500 mM. Other weak acids or their salts can also be used.

The composition may also include solubilizing agents, preservatives, stabilizers, emulsifiers, and the like. A local anesthetic (e.g., lidocaine) may also be included in the compositions, particularly for injectable forms, to ease pain at the site of the injection.

Effective amounts of the peptide will depend on the nature of use, including the nature of the cancerous condition which is being treated, tumor volume and stage, and its location(s). By way of example only, suitable peptide concentrations may range from about 1 μM to about 10 mM, preferably about 10 μM to about 5 mM, about 50 μM to about 2 mM, or about 100 μM to about 1 mM. The volume of the composition administered, and thus, dosage of the peptide administered can be adjusted by one of skill in the art to achieve optimized results. By way of example, 250 μg to 2000 μg can be administered per day, repeated periodically as needed, e.g., every third day, once weekly, every other week, etc. This can be adjusted lower to identify the minimal effective dose, or tailored higher or lower according to the nature of the tumor to be treated.

Further aspects of the invention relate to methods of forming a nanofibril network on or near the surface of target cells; methods of treating a cancerous condition in a patient; methods of in vivo imaging, and methods of collecting a target cell secretome.

The method for forming a nanofibril network on or near the surface of target cells includes the step of contacting a target cell that expresses a cell surface-bound enzyme having hydrolytic activity, secretes an enzyme having hydrolytic activity, or both, with a phospho-aryl peptide of the invention or a pharmaceutical composition of the invention, wherein the contacting is effective to hydrolyze the phosphate group and cause in situ self-assembly of the peptides to form a nanofibril network on or near the surface of the target cell. The target cell expresses a cell surface-bound phosphatase, secretes a phosphatase, or both.

When performed ex vivo or surgically recovered from an individual subsequent to an in vivo treatment in accordance with the present invention, the nanofibril network which sequesters or contains cell signaling molecules can be harvested and used independently either for raising therapeutic antibodies (a passive anti-cancer vaccine component) or as a component in an active anti-cancer vaccine formulation.

In each of the above embodiments relating to methods of forming a nanofibril network on or near the surface of target cells, the target cells may be cancer cells. In accordance with these embodiments of the method of forming a nanofibril network on or near the surface of target cells, the nanofibril network results in a gel outside cells to sequester cell signaling molecules, wherein the cell signaling molecules are from cancer cells or from a cancer microenvironment. Because the nanofibril network retains the target cell secretome from the pericellular space of the target cell, the nanofibril network containing the secretome can be recovered, and the target cell secretome separated from the nanofibril network and collected. Recovery of the nanofibril network can be carried out using cold shock to detach the nanofibril network from the target cells, followed by centrifugation.

In certain embodiments, the gel containing the cancer cell signaling molecules can be used for raising antibodies against cancers. In additional embodiments, the contacting is effective to inhibit cancer cell migration, inhibit cancer cell survival, inhibit cancer cell growth, and/or inhibit passage of intracellular signaling molecules to or from the nanofibril network-covered cancer cell.

The method of treating a cancerous condition in a subject includes the step of administering to a subject having a cancerous condition a therapeutically effective amount of a phospho-aryl peptide of the invention or a pharmaceutical composition of the invention, wherein the administering is effective to cause in vivo self-assembly of the peptides to form a nanofibril network on or near the surface of cancer cells, which has the effects noted above. Exemplary subjects include any mammal that is susceptible to cancerous conditions including, without limitation, rodents, rabbits, canines, felines, ruminants, and primates such as monkeys, apes, and humans.

Administration of the phospho-aryl peptide or pharmaceutical composition can be carried out using any suitable approach. By way of example, administration can be carried out parenterally, subcutaneously, intravenously, intradermally, intramuscularly, intraperitoneally, by implantation, by intracavitary or intravesical instillation, intraarterially, intralesionally, intradermally, peritumorally, intratumorally, or by introduction into one or more lymph nodes. In certain embodiments, administration is carried out intralesionally, intratumorally, intradermally, or peritumorally.

In certain embodiments, the peptide is conjugated with a chemotherapeutic agent, an antiangiogenic agent, an immunomodulating agent, or an antigen. In one embodiment, the peptide may be conjugated with a thermoablative nanoparticle.

In these several aspects of the invention, the cancer cells express a cell surface-bound phosphatase, secrete a phosphatase, or both.

The cancer cells to be treated in accordance with these aspects can be present in a solid tumor, present as a metastatic cell, or present in a heterogenous population of cells that includes both cancerous and noncancerous cells. Exemplary cancer conditions include, without limitation, cancers or neoplastic disorders of the brain and CNS (glioma, malignant glioma, glioblastoma, astrocytoma, multiforme astrocytic gliomas, medulloblastoma, craniopharyngioma, ependymoma, pinealoma, hemangioblastoma, acoustic neuroma, oligodendroglioma, meningioma), pituitary gland, breast (Infiltrating, Pre-invasive, inflammatory cancers, Paget's Disease, Metastatic and Recurrent Breast Cancer), blood (Hodgkin's Disease, Leukemia, Multiple Myeloma, Lymphoma), lymph node cancer, lung (Adenocarcinoma, Oat Cell, Non-small Cell, Small Cell, Squamous Cell, Mesothelioma), skin (melanoma, basal cell, squamous cell, Kapsosi's Sarcoma), bone cancer (Ewing's Sarcoma, Osteosarcoma, Chondrosarcoma), head and neck (laryngeal, pharyngeal, and esophageal cancers), oral (jaw, salivary gland, throat, thyroid, tongue, and tonsil cancers), eye, gynecological (Cervical, Endrometrial, Fallopian, Ovarian, Uterine, Vaginal, and Vulvar), genitourinary (Adrenal, bladder, kidney, penile, prostate, testicular, and urinary cancers), and gastrointestinal (appendix, bile duct (extrahepatic bile duct), colon, gallbladder, gastric, intestinal, liver, pancreatic, rectal, and stomach cancers).

Use of the phospho-aryl peptides and pharmaceutical compositions can be coordinated with previously known therapies. For instance, where the phospho-aryl peptide is conjugated with a thermoablative nanoparticle, after formation of the pericellular nanofibril network, a tumor-containing region of the subject's body can be exposed to near infrared light, thereby causing thermal heating of the thermoablative nanoparticle and destruction of cancer cells covered by the nanofibril network.

In addition, chemotherapeutic agents, immunotherapeutic agents, or radiotherapeutic agents, as well as surgical intervention can be used in a coordinated manner with the phospho-aryl peptides or pharmaceutical compositions of the present invention. Thus, a chemotherapeutic agent, an immunotherapeutic agent, or a radiotherapeutic agent can be administered to a patient before or after treatment with the phospho-aryl peptide or pharmaceutical compositions of the present invention. Alternatively, surgical resection of a tumor can be carried out before or after treatment with the phospho-aryl peptides or pharmaceutical compositions of the present invention.

### EXAMPLES

The following Examples are presented to illustrate various aspects of the disclosure, but are not intended to limit the scope of the claimed invention.

### Materials and Methods for Examples 1-4

Materials: 2-Cl-trityl chloride resin (1.0-1.2 mmol/g) and Fmoc-d-phenylalanine were obtained from GL Biochem (Shanghai, China). 4′-hydroxy-[1,1′-biphenyl]-4-carboxylic acid was obtained from 1PlusChem. 4-hydroxybenzoic acid was obtained from ACROS Organics. 6-hydroxy-2-naphthoic acid was obtained from Sigma-Aldrich. Other chemical reagents and solvents were obtained from Fisher Scientific; all chemical reagents and solvents were used as received from commercial sources without further purification; alkaline phosphatase was purchased from Biomatik.

Instruments: All precursors were purified with Agilent 1100 Series HPLC system equipped with a reverse phase C18 column. LC-MS was conducted on a Waters Acquity Ultra Performance LC with Waters MICRO-MASS detector. Rheology tests were obtained by a TA ARES-G2 rheometer at 25° C. TEM images were taken on a Morgagni 268 transmission electron microscope. SEM images were taken on a JEOL JSM-6060LV SEM model with an accelerating voltage of 15 kV.

Hydrogelation Experiment: Gelation experiments were carried out in 1.5 mL glass vials. 1 mg of each of the six precursors was first dissolved in 100 μL of 1×PBS buffer. The pH of the solution was carefully adjusted to 7.4 with 1 M NaOH (aq). Then extra PBS buffer was added to make the solutions with the volume of 200 μL and final concentration of 0.5 wt %. 1 μL of 0.2 U μL⁻¹ ALP was added to make the final concentration of 1 U mL⁻¹. After incubation at room temperature for 24 h, the hydrogel was formed.

Rheology Experiment: Rheological tests were conducted on TA ARES-G2 rheometer, parallel-plate geometry with an upper plate diameter of 25 mm was used during the experiment, and the gap was 1 mm. Solutions of 3P, 5P and 7P were made with the concentration of 8 mM and volume of 400 μL. Next, ALP was added to the solutions and mixed with a pipette. Then, the samples were quickly loaded onto the stage, and oscillation time-dependent strain was performed: 1.0%, frequency: 1 Hz), strain sweep (0.1-100%) at 6.28 rad/s, frequency sweep test (0.1-200 rad/s).

TEMSample Preparation: Sample solution was placed on the TEM grid (5 μL, sufficient to cover the grid surface). Approximately ˜10 sec later, sample rinsing was carried out by placing a large drop of the ddH₂O on parafilm and the grid was allowed to touch the water drop, with the sample-loaded surface facing the parafilm. Tilting the grid and gently absorbing water from the edge of the grid using a filter paper sliver. This rinsing process was carried out 3 times. Immediately after rinsing, staining was carried by placing a large drop of the uranyl acetate (UA) stain solution on parafilm and allowing the grid touch the stain solution drop, with the sample-loaded surface facing the parafilm. Tilting the grid and gently absorbing the stain solution from the edge of the grid using a filter paper sliver ensured full coverage. The grid was allowed to dry in air, and the dried grids were examined as soon as possible.

SEM Sample Preparation: The morphologies of the xerogels were characterized using scanning electron microscopy (SEM-JEOL JSM-6060LV) operating with an accelerating voltage of 5-30 kV. The xerogels were prepared by drying in an oven at 70° C. overnight. To minimize charging, the samples were coated with a thin layer of gold before the experiment.

CMC Measurement: A series of 2P/3P solutions from the concentration of 4 mM to 0.25 M was prepared in pH 7.4 PBS buffer. After incubating with Rhodamine 6G (5 M), the λmax was determined by measuring the absorbance from 520 to 540 nm using a Biotek Synergy 4 hybrid multi-mode microplate reader.

Cell Culture: Saos2, SJSA1 and HepG2 cells were purchased from American Type Culture Collection (ATCC, USA). Saos2 cells were cultured in McCoy's 5A Medium (Gibco, Life Technologies) supplemented with 15% (v/v) fetal bovine serum (FBS) (Gibco, Life Technologies), 100 U/mL penicillin and 100 g/mL streptomycin (Gibco, Life Technologies); SJSA1 cell were culture in RPMI1640 (ATCC, USA) Medium supplemented with 10% (v/v) FBS, 100 U/mL penicillin and 100 g/mL streptomycin; HepG2 cells were cultured in Minimal Essential Medium (MEM) (Gibco, Life Technologies) supplemented with 10% (v/v) FBS, 100 U/mL penicillin and 100 g/mL streptomycin. All the cells were maintained at 37° C. in a humidified atmosphere of 5% CO₂.

MTT Assay: Cells were seeded in 96-well plates at 1×10⁴ cells/well for 24 hours to allow attachment. After removing the culture medium, fresh culture medium containing different concentration of the precursors were added. After 24/48/72 hours, 10 μL MTT (ACROS Organics) solution (5 mg/mL) was added to each well to incubate at 37° C. for 4 h. 100 μL of SDS-HCl solution was then added to stop the reduction reaction and dissolve the formazan. The absorbance of each well at 595 nm was measured by a DTX880 Multimode Detector. The results were calculated as cell viability percentage relative to untreated cells. The MTT assay was performed in triplet (n=3) and the average value of the three measurements was taken.

For cell death rescue experiment, after cell attachment, the cells were pretreated with TNAP inhibitors or other cell death inhibitors for 30 min, and then co-cultured with the mixture of 2P/3P with different inhibitors. After 2 hours, same procedures are carried out to get the cell viability percentage relative to untreated cells.

Confocal Microscopy: Saos2 and SJSA1 cells were seeded at 1.5×10⁵ cells in a 3.5 cm confocal dish for 24 hours to allow attachment. Following the removal of culture medium, fresh culture medium containing 20 M of 4P was added. After 0.5 h of incubation, cells were washed with live cell image solution (Life Technologies A14291DJ) 3 times and stained with 1× ER-Tracker Red, Lyso tracker deep red, and Mito tracker deep red (Invitrogen) in live cell imaging solution. Finally, the cells were kept in the live cell imaging solution for imaging using Zeiss LSM 880 confocal microscopy at the lens of 63× with oil.

### Example 1—Synthesis of Peptides 2P-7P

Preparation of intermediates phosphophenyl carboxylic acid (ₚB), phosphonaphthoic acid (ₚNP), and phosphobiphenyl carboxylic acid (ₚBP) was first carried out prior to solid phase synthesis. ₚB, ₚNP and ₚBP were prepared according to previously reported procedures (Graber et al., ACS Chemical Biology 6:1008 (2011), which is hereby incorporated by reference in its entirety). Briefly, 4-hydroxybenzoic acid, 6-hydroxy-2-naphthoic acid, or 4′-hydroxy-[1,1′-biphenyl]-4-carboxylic acid (1 equivalent) and PCl₅ (1 equivalent) were stirred at room temperature for 45 min. Subsequently, the reaction was brought to completion by sonication at 60° C. for 90 min. The ice-cooled reaction mixture was dissolved in 10 mL of acetone and 10 mL of benzene, and 1.4 mL (3 equivalent) of distilled water was added dropwise. After stirring at 0° C. for 30 min, 20 mL benzene was added. The reaction mixture was stirred at room temperature for 12 h. The precipitate was filtered off, washed with 20 mL benzene, and dried in high vacuum.

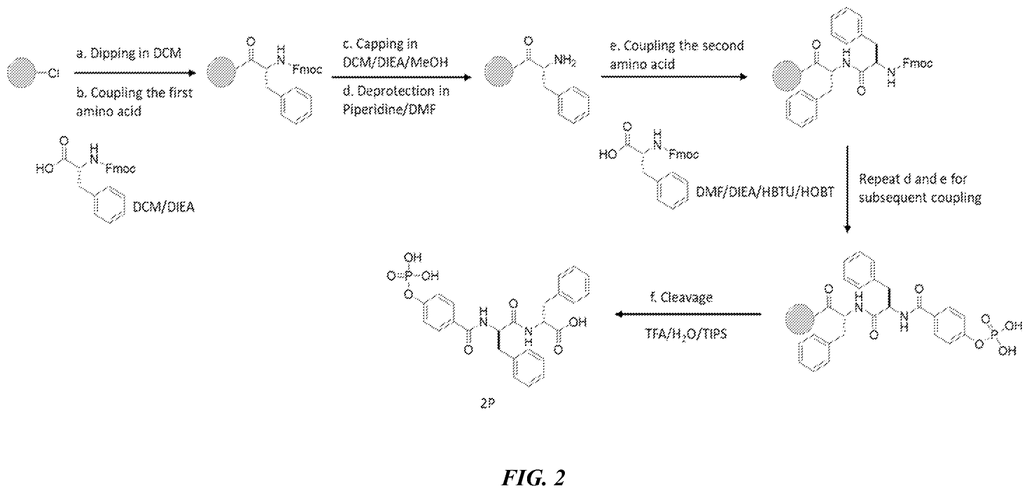

Using Fmoc-based solid-phase peptide synthesis (Chan et al., Fmoc Solid Phase Peptide Synthesis: A Practical Approach, OUP Oxford (1999), which is hereby incorporated by reference, synthesis of peptides 2P-7P was carried out as illustrated in FIG. 2. Briefly, 2-chlorotrityl chloride resin was weighed and dipped in methylene chloride (DCM) for 15 min. The amino acids were weighed according to 1.2 mmol/g of resin. The amino acid was dissolved in DCM with the addition of 2.5 equivalent of N, N-Diisopropylethylamine (DIEA), then the resin was mixed with the solution well on a rocker for 1 h, and then washed with DCM. Capping solution (DCM:MeOH:DIEA=17:2:1) was added to the well and allowed to react for 15 min. Washing was carried out using DCM first and then dimethylformamide (DMF). 20% piperidine in DMF for 30 min was sufficient to remove Fmoc group, followed by washing with DMF. Next, the amino acid (1 equivalent), HBTU (1 equivalent), and DIEA (2.5 equivalent) was loaded in DMF for 40 min and subsequently washed with DMF. Finally, DCM was used to wash out DMF. Peptide cleavage from the resin was achieved using 95% TFA, 2.5% triisopropyl silane (TIPS), 2.5% H₂O for 30 min. After that, the solvent is air dried and the remained oily product was precipitated out by adding ethyl ether and filtered to recover the peptide. Then the peptide was dissolved in methanol and purified by RP-HPLC to obtain the purified peptide products.

The structure of peptides 2P-7P was confirmed by LC-MS.

### Example 2—Synthesis of O-Methyl Peptide Derivatives

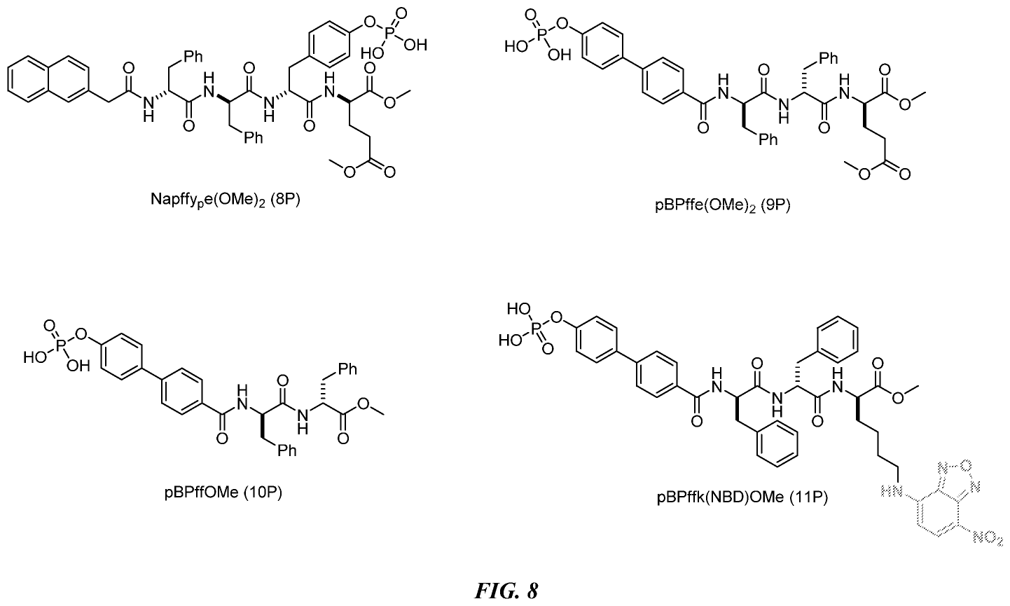

Using the peptide 6P (pBP-ff), an O-methyl derivative, 10P (FIG. 8), was prepared as follows. pBP-ff (1 equivalent) was dissolved in DCM with stirring and then bromotrimethylsaline (15 equivalent) was added. The reaction mixture was stirred at room temperature overnight. After air drying, methanol was added, and the reaction was stirred at room temperature for 1 day. Then the product was purified with HPLC.

The same procedures were used to prepare the O-methyl variant of peptide pBP-ffe, 9P (FIG. 8), which is methylated on the glutamic acid sidechain as well as the C-terminal carboxylic acid group; and the O-methyl variant of peptide pBP-ffk(NBD), 11P (FIG. 8), which is methylated on the C-terminal carboxylic acid group and includes the NBD fluorophore tethered to the lysine sidechain. The control peptide bearing a naphthyl N-terminal capping group and a phosphorylated D-Tyr residue, Nap-ffyₚe-(OMe)₂, 8P (FIG. 8), was prepared using solid phase synthesis and methylation in the same manner.

### Example 3—Evaluation of Enzymatic Self-Assembly Using Peptides 2P-7P as Substrates

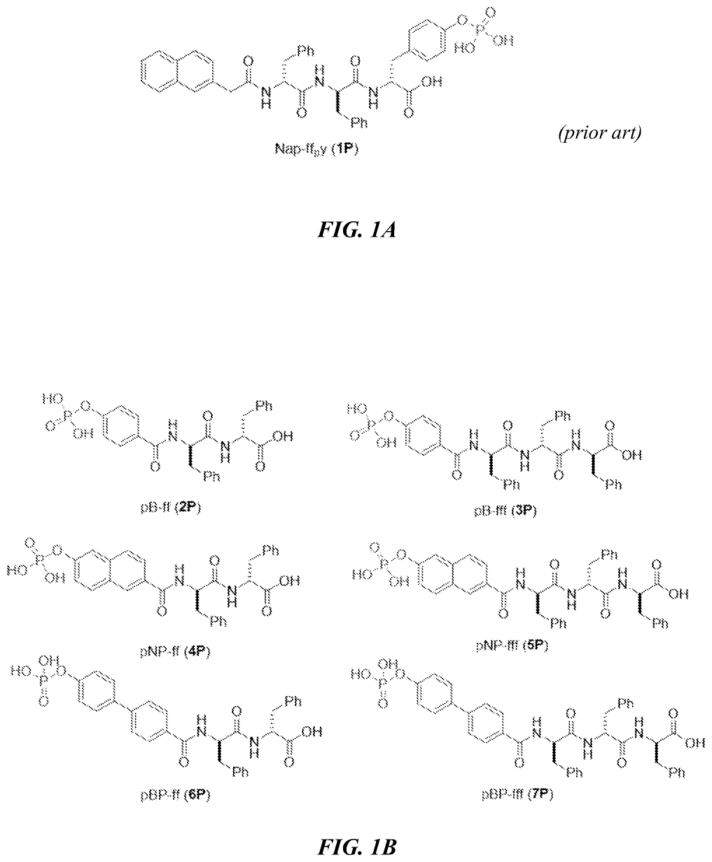

The dephosphorylation of N-terminal aromatic capping motifs of short peptides was examined for enzymatic self-assembly and hydrogelation. As shown in FIG. 1B, three different N-terminal aromatic capping motifs were examined on two related D-peptides: phosphohydroxybenzoic acid (ₚB), phosphohydroxynaphthoic acid (ₚNP), and phosphohydroxybiphenyl-carboxylic acid (ₚBP) at the N-terminal of D-diphenylalanine (ff) or D-tri-phenylalanine peptide (fff) to generate phosphorylated peptide derivatives (2P-7P) as the substrates of ALP. Based on the structure of 1P (FIG. 1A), ₚB, ₚNP or pBP were used as the enzymatic trigger of ALP to replace ₚy in 1P. The ALP trigger was also moved from the C-terminal end in 1P to the N-terminal end of the peptides. That is, ₚB, ₚNP or ₚBP act as the N-terminal capping group for ff or fff. This combination leads to six substrates of ALP: ₚB-ff (2P), PB-fff (3P), ₚNP-ff (4P), ₚNP-fff (5P), pBP-ff (6P), and pBP-fff (7P). After being dephosphorylated by ALP, these substrates result in six peptide derivatives: B-ff (2), B-fff (3), NP-ff (4), NP-fff (5), BP-ff (6), and BP-fff (7).

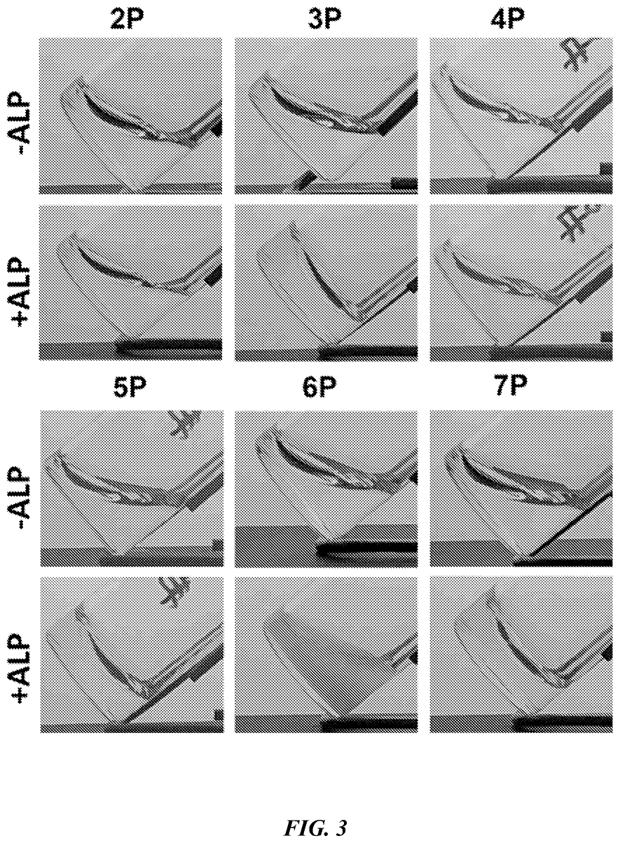

The enzymatic gelation of the phosphorylated peptide derivatives (2P-7P) was evaluated upon the addition of ALP. Each of precursor dissolves in PBS buffer to form a clear solution with the concentrations of 0.5 wt %. As shown in FIG. 3, enzymatic dephosphorylation of 3P, 5P or 7P results in a hydrogel 24 h after adding ALP. While the dephosphorylation of 2P and 4P affords a solution, the dephosphorylation of 6P results in a suspension. This result indicates that 2 and 4 are more water soluble than 6, agreeing with the higher hydrophobicity of biphenyl (log P=3.71) than those of naphthyl (log P=3.03) and phenyl (log P=2.03) groups. These results also indicate that the tri-phenylalanine enhances intermolecular interactions to favor hydrogelation of 3, 5, or 7.

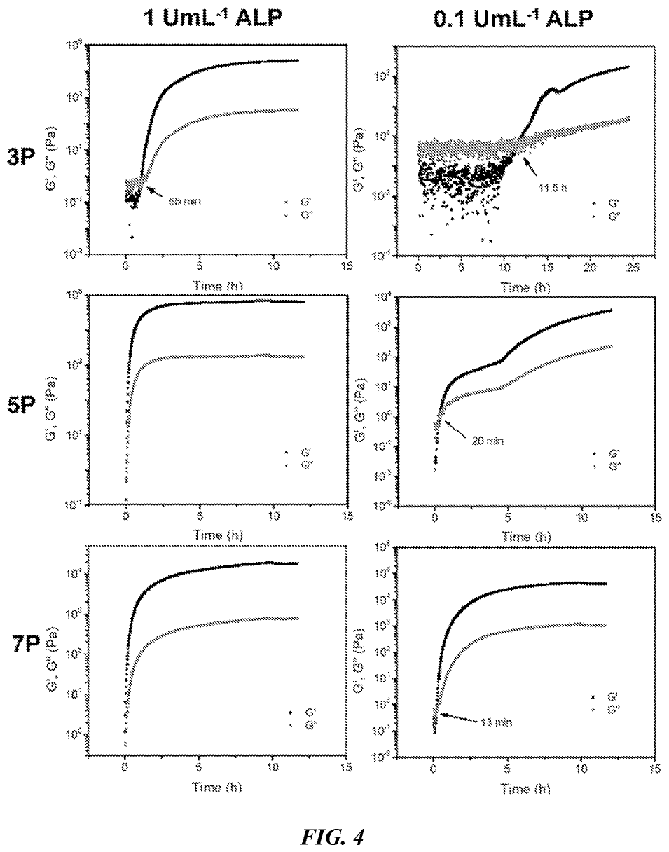

Next, a dynamic time sweep was used to characterize the rheological properties of the hydrogels resulted from EISA of 3P, 5P, and 7P by measuring their storage and loss moduli (G′ and G″) (FIG. 4). The solutions of 3P, 5P, and 7P were made with the concentrations of 8 mM (about 0.5 wt %). After the addition of ALP to the solution of 3P, a crossover of G′ and G″ occurs at about 1 h or 11 h of incubation when the concentration of ALP is 1.0 or 0.1 UmL⁻¹, respectively. With the treatment of 1.0 UmL⁻¹ or 0.1 UmL⁻¹ ALP, the solution of 5P shows the crossover of G′ and G″ around 2 minutes or at 20 minutes, respectively. Being incubated with ALP at 1.0 or 0.1 UmL⁻¹, the solution of 7P exhibits the crossover of G′ and G″ less than one minute or at about 13 minutes, respectively. These results indicate that ₚBP or ₚNP, as an enzyme trigger, enables enzymatic hydrogelation about 50-60 times faster than ₚB. Moreover, the times of 5P and 7P to reach the gelation point are roughly two to three times shorter than that of 1P. This improvement was not expected. This result confirms that ₚBP and ₚNP are faster enzyme triggers for EISA than ₚy present in 1P. After 12 hours, the G′ values of the hydrogels by EISA of 3P, 5P, and 7P reach their plateau values when the concentration of ALP is 1.0 UmL⁻¹.

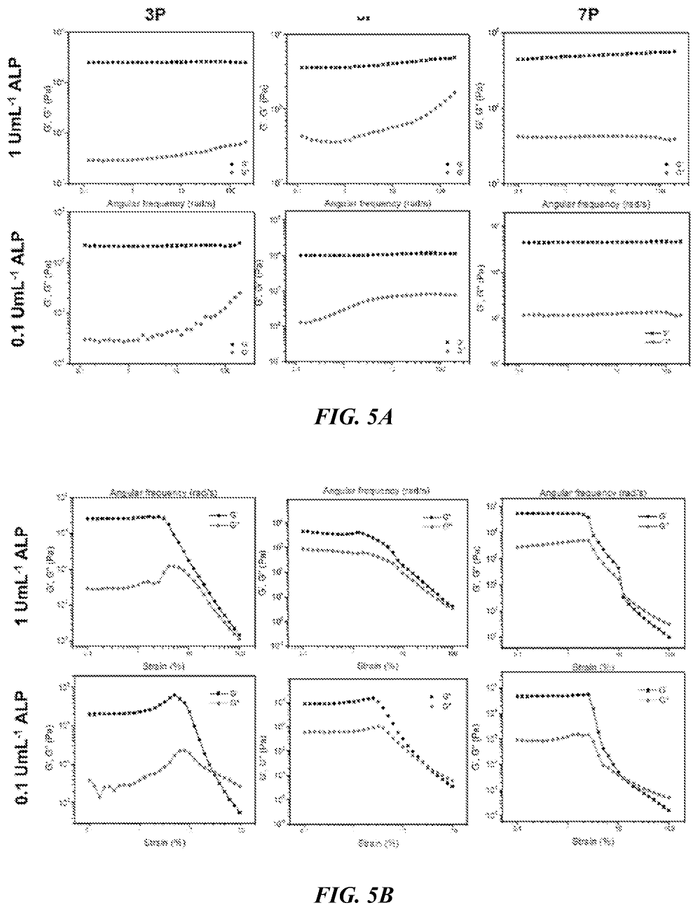

Frequency and strain sweeps of the hydrogels were also performed (FIGS. 5A-5B). The strain and frequency applied in time sweep fall within the linear viscoelastic range of the gels, indicating that the time-dependent strain sweeps are carried out under appropriate conditions. Being incubated with 1 UmL⁻¹ and 0.1 UmL⁻¹ of ALP, the hydrogels, being made of 3, 5, or 7, obtained by the dephosphorylation of 3P, 5P, or 7P, exhibit frequency-independent G′ (0.1 rads⁻¹ to 200 rads⁻¹), suggesting that gels behave solid-like. For the gel resulting from dephosphorylation of 3P by 1 UmL⁻¹ of ALP, G′ and G″ are independent of strain below 1% and show the existence of linear viscoelastic region (LVR). Within the LVR, G′ (up to 10⁴ Pa) is significantly greater than G″, reflecting their dominant elastic nature. For the gel resulting from dephosphorylation of 3P by 0.1 UmL⁻¹ of ALP, though G′>G″ below 1% strain, G″ fluctuates and increases with the increase of strain, which fails to show LVR and indicates the hydrogel is relatively weak. Unlike the case of 3P, the strain sweeps of the hydrogels resulting from dephosphorylation of 5P or 7P by 1 or 0.1 UmL⁻¹ of ALP show that G′ and G″ are independent of strain below 2%. Both show the existence of linear viscoelastic region (LVR), suggesting that NP or BP enhances the viscoelasticity of the hydrogels made of 5 or 7.

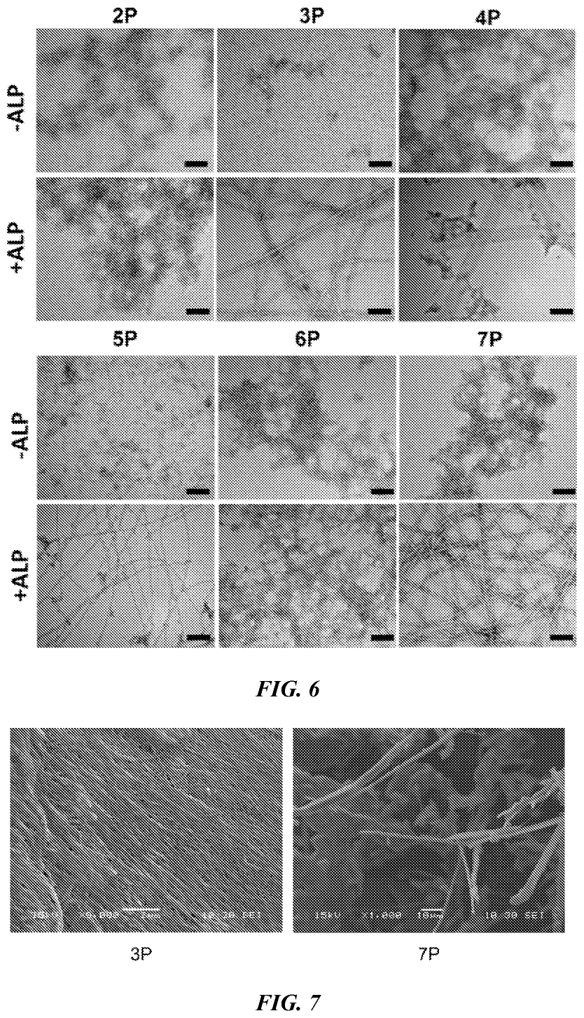

To investigate the morphological properties, transmission electron microscopy (TEM) was used to image these precursors without or with the addition of ALP. As shown in FIG. 6, TEM of the solutions of 2P, 4P, 6P, and 7P show aggregated nanoparticles, with the diameters about 10 nm. While the TEM of the solution of 3P reveals the existence of short nanofibers with the diameters of 4 nm, TEM of the solutions of 5P show the coexistence of short nanofibers with diameters of 4 nm and nanoparticles. After the addition of 0.1 UmL⁻¹ of ALP into the solutions of 2P and 6P for 24 h, the resulting solution of 2 and the suspension of 6 contain the nanoparticles with the diameters around 12 nm. The resulting solution of 4 showed coexistence of nanoparticles and nanosheets. The hydrogels of 3 show extended and entangled nanofibers with the diameters of 4 nm, and some of the nanofibers form bundles with a diameter of 14 nm; the hydrogel of 5 shows uniform nanofibers with a diameter of 8 nm; and the hydrogel of 7 shows uniform bundles with a diameter of ˜13±2 nm. The formation of the nanofibers of 3, 5, or 7 likely contributes to their formation of hydrogels. Notably, while the scanning electron microscopic (SEM) image of the dried gel of 3 display nanofiber networks, the dried gel of 7 is largely amorphous with a few thick fibres (FIG. 7), agreeing with that the high hydrophobicity of biphenyl group significantly enhances the intermolecular interactions of 7.

In summary, six short peptides were designed to contain a phosphoaromatic as both the capping groups and the enzyme trigger at the N-terminal, and are demonstrated to be novel ALP substrates for EISA and hydrogelation. The ability to form the hydrogels indicate that the tripeptide backbone having aromatic groups (i.e., Phe and/or Tyr) enhances self-assembly and leads to hydrogelation. The result that ₚBP is a faster substrate than pTyr for ALP agrees with the report that ₚBP is a faster substrate than pTyr for protein tyrosine phosphatase 1 (PTP1B) (Montserat et al., J Biol Chem 271:7868 (1996), which is hereby incorporated by reference in its entirety). The rates of the enzymatic hydrogelation catalysed by ALP follows the trend of 7P>5P>1P>3P, implying that distancing the phosphate trigger away from the peptide backbone likely favors fast enzymatic self-assembly, a design principle that may help combine EISA with other self-assembling molecules (Hamley, Soft Matter 7:4122 (2011); Hamley et al., Soft Matter 9:9290 (2013); Shi et al., Angew Chem Int Ed 57:11188 (2018); Wu et al., Nature 574:658 (2019); Castelletto et al., Chem Commun 56:11977 (2020); Gayen et al., Soft Matter 16:10106 (2020); Jervis et al., Soft Matter 16:10001 (2020); Liu et al., Soft Matter 16:4115 (2020); Yoshisaki et al., Chem Asian J 16:1937 (2021), each of which is hereby incorporated by reference in its entirety). This work also supports the expectation that other phospho-bis- and phospho-tris-aromatic capping groups can be used to develop EISA-catalyzed anticancer drug candidates that act via by phosphatases.

### Example 4—Evaluation of Anti-Cancer Activity of Phospho-Peptides and O-Methyl Variants

Saos-2 is a human osteosarcoma cell line, which displays several osteoblastic features and is known to have high basal alkaline-phosphatase activity. SJSA-1 is a human osteosarcoma cell line that has demonstrated greater metastatic potential than Saos-2. HepG2 is a human liver cancer cell line that expresses alkaline-phosphatase. Saos-2, SJSA-1, and HepG2 were used for cell-based assays to evaluate the activity of various peptides and O-Methyl variants.

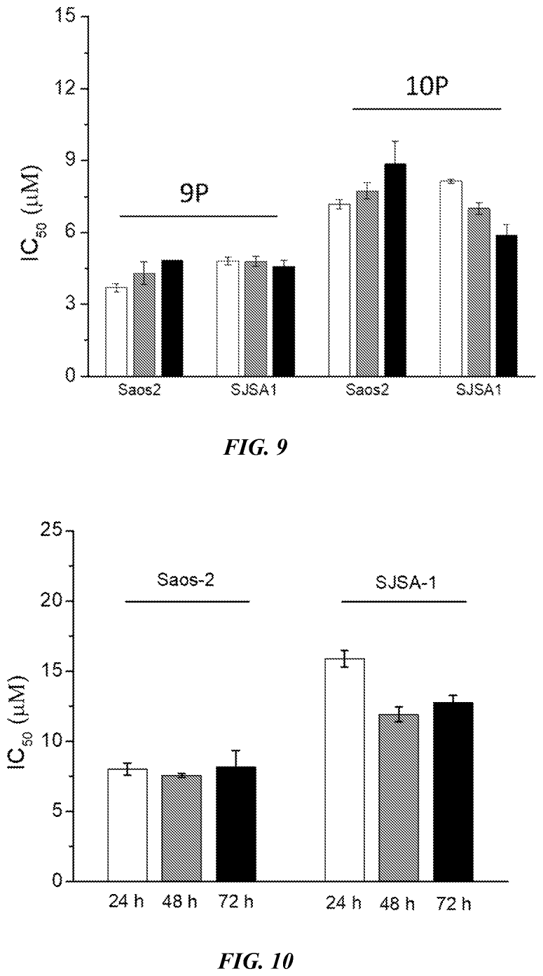

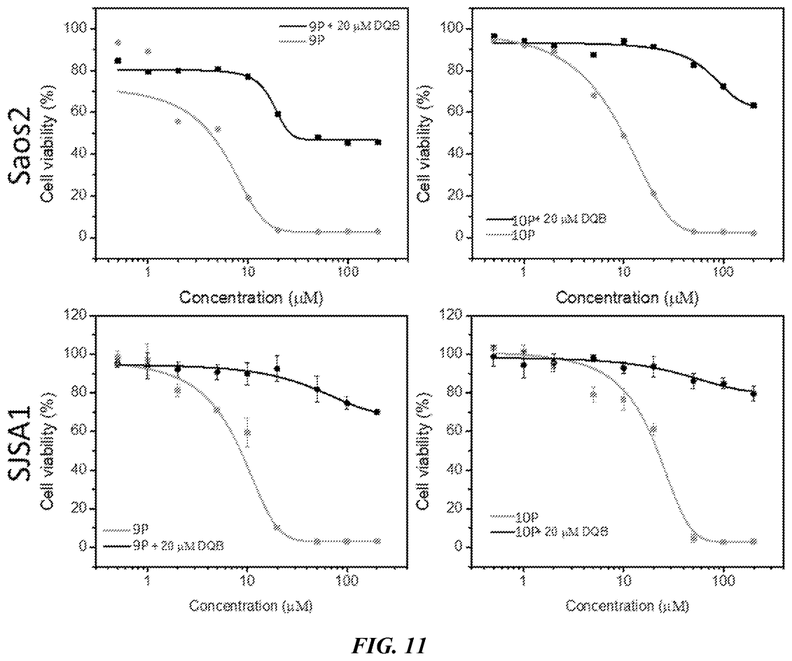

FIGS. 9 and 10 together illustrate the IC₅₀ values of 9P-11P incubated with Saos2 and SJSA1 cells at 24 h, 48 h, and 72 h. As shown in FIGS. 9 and 10, 9P-11P displayed a low micromolar IC₅₀ against Saos2 and SJSA-1 cells. Of these three peptides, 9P displayed the lowest IC₅₀ values (˜3-4 μM). FIG. 11 illustrates the cell viability curves for varying concentrations of 9P and 10P Saos2 and SJSA-1 cells in the presence or absence of DQB (20 μM). The duration time was 2 h. 9P displayed a higher cytotoxicity to Saos2 and SJSA1 when compared to 10P.

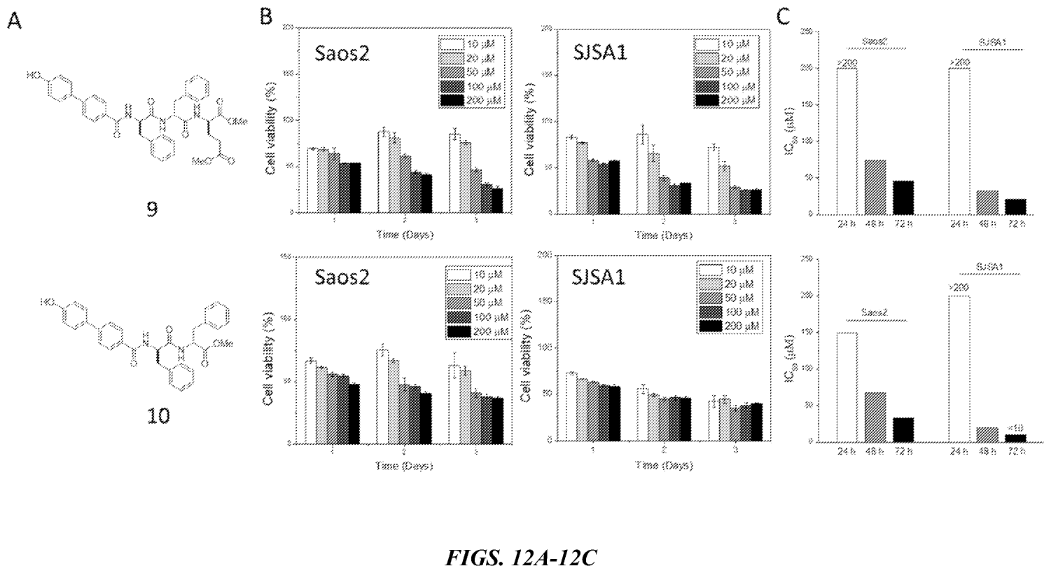

FIG. 12A illustrates the structures of dephosphorylated peptides 9 and 10. FIG. 12B illustrates the cell viability of Saos2 and SJSA1 treated with peptides 9 and 10, and FIG. 12C illustrates the IC₅₀ value of 9 and 10 incubated with Saos2 and SJSA1.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A peptide comprising from 3 to 20 amino acids, including at least two aromatic amino acid residues and an N-terminal phosphorylated aryl group, wherein upon exposure to an enzyme that hydrolyzes the phosphate group the peptide self-assembles to form nanofibrils and optionally nanoparticles.

2. The peptide according to claim 1, wherein the aromatic amino acids are selected from the group consisting of phenylalanine, phenylalanine derivatives, napthylalanine, napthylalanine derivative, tyrosine, tyrosine derivatives, tryptophan, and tryptophan derivatives.

3. The peptide according to claim 1, wherein the amino acids are all D-amino acids or all L-amino acids.

4. The peptide according to claim 1, wherein the amino acids are a mixture of L-amino acids and D-amino acids.

5. The peptide according to claim 1, wherein the phosphorylated aryl group is a phosphobisaromatic group or phosphotrisaromatic group.

6. The peptide according to claim 5, wherein the phosphobisaromatic group is 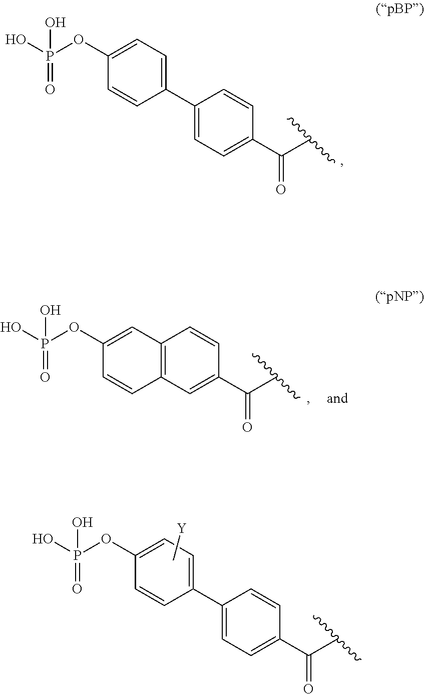
where Y is —F, —Cl, —Br, or —CN.

7. The peptide according to claim 5, wherein the phosphotrisaromatic group is 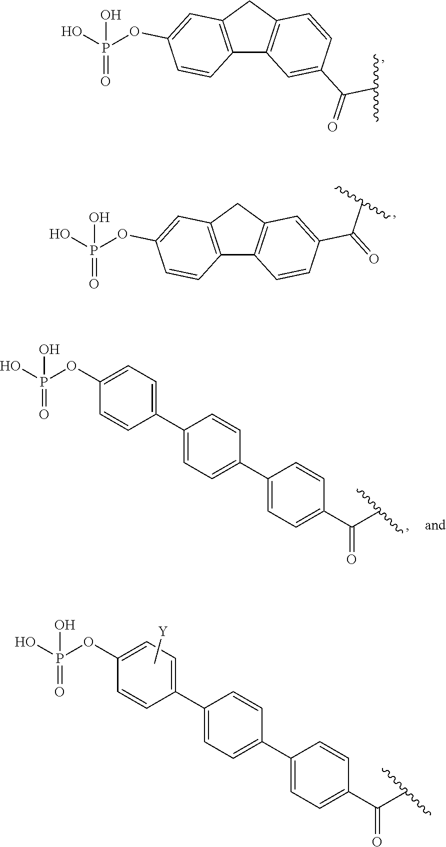
where Y is —F, —Cl, —Br, or -CN.

8. The peptide according to claim 1, wherein the peptide further comprises a fluorophore, a chemotherapeutic agent, an antiangiogenic agent, a thermoablative nanoparticle, an immunomodulating agent, or an antigen coupled to a C-terminus of the peptide.

9. The peptide according to claim 1, wherein the peptide further comprises a fluorophore, a chemotherapeutic agent, an antiangiogenic agent, a thermoablative nanoparticle, an immunomodulating agent, or an antigen coupled to a sidechain of an amino acid residue.

10. The peptide according to claim 9, wherein the amino acid residue is lysine, arginine, or cysteine.

11. The peptide according to claim 1, wherein said peptide is up to about 10 amino acids.

12. (canceled)

13. The peptide according to claim 1, wherein at least 40% of the amino acid residues in the peptide are aromatic amino acid residues.

14. The peptide according to claim 1, wherein the peptide comprises an amino acid sequence of fff or FFF, ffff or FFFF (SEQ ID NO:1), ffkf or FFKF (SEQ ID NO:2), ffky or FFKY (SEQ ID NO:3), ffyk or FFYK (SEQ ID NO:4), fffk or FFFK (SEQ ID NO:5), fffff or FFFFF (SEQ ID NO:6), ffgff or FFGFF (SEQ ID NO:7), fffgf or FFFGF (SEQ ID NO:8), ffffg or FFFFG (SEQ ID NO:9), ffe or FFE, fffe or FFFE (SEQ ID NO:10), ffke or FFKE (SEQ ID NO:11), ffek or FFEK (SEQ ID NO:12), ffffe or FFFFE (SEQ ID NO:13), ffeff or FFEFF (SEQ ID NO:14), fffef or FFFEF (SEQ ID NO:15), ffd or FFD, fffd or FFFD (SEQ ID NO:16), ffkd or FFKD (SEQ ID NO:17), ffdk or FFDK (SEQ ID NO:18), ffffd or FFFFD (SEQ ID NO:19), ffdff or FFDFF (SEQ ID NO:20), fffdf or FFFDF (SEQ ID NO:21), nal-fff or Nal-FFF (SEQ ID NO:22), nal-ffff or Nal-FFFF (SEQ ID NO:23), nal-ffkf or Nal-FFKF (SEQ ID NO:24), nal-ffky or Nal-FFKY (SEQ ID NO:25), nal-ffyk or Nal-FFYK (SEQ ID NO:26), nal-fffk or Nal-FFFK (SEQ ID NO:27), nal-fffff or Nal-FFFFF (SEQ ID NO:28), nal-ffgff or Nal-FFGFF (SEQ ID NO:29), nal-fffgf or Nal-FFFGF (SEQ ID NO:30), nal-ffffg or Nal-FFFFG (SEQ ID NO:31), nal-ffe or Nal-FFE (SEQ ID NO:32), nal-fffe or Nal-FFFE (SEQ ID NO:33), nal-ffke or Nal-FFKE (SEQ ID NO:34), nal-ffek or Nal-FFEK (SEQ ID NO:35), nal-ffffe or Nal-FFFFE (SEQ ID NO:36), nal-ffeff or Nal-FFEFF (SEQ ID NO:37), nal-fffef or Nal-FFFEF (SEQ ID NO:38), nal-ffd or Nal-FFD (SEQ ID NO:39), nal-fffd or Nal-FFFD (SEQ ID NO:40), nal-ffkd or Nal-FFKD (SEQ ID NO:41), nal-ffdk or Nal-FFDK (SEQ ID NO:42), nal-ffffd or Nal-FFFFD (SEQ ID NO:43), nal-ffdff or Nal-FFDFF (SEQ ID NO:44), and nal-fffdf or Nal-FFFDF (SEQ ID NO:45),
wherein lowercase letters denote D-amino acids, uppercase letters denote L-amino acids, nal is a D-naphthylalanine (3-(I-Naphthyl)-D-alanine or 3-(2-Naphthyl)-D-alanine), and Nal is an L-naphthylalanine (3-(I-Naphthyl)-L-alanine or 3-(2-Naphthyl)-L-alanine).

15. The peptide according to claim 1, wherein the peptide is selected from the group consisting of:
X₁-fff-Z₁ or X₁-FFF-Z₁, X₁-ffff-Z₁ or X₁-FFFF-Z₁ (SEQ ID NO:46), X₁-ffkf-Z₁ or X₁-FFKF-Z₁ (SEQ ID NO:47), X₁-ffky-Z₁ or X₁-FFKY-Z₁ (SEQ ID NO:48), X₁-ffyk-Z₁ or X₁-FFYK-Z₁ (SEQ ID NO:49), X₁-fffk-Z₁ or X₁-FFFK-Z₁ (SEQ ID NO:50), X₁-fffff-Z₁ or X₁-FFFFF-Z₁ (SEQ ID NO:51), X₁-ffgff-Z₁ or X₁-FFGFF-Z₁ (SEQ ID NO:52), X₁-fffgf-Z₁ or X₁-FFFGF-Z₁ (SEQ ID NO:53), X₁-ffffg-Z₁ or X₁-FFFFG-Z₁ (SEQ ID NO:54), X₁-ffk(Z₂)f or X₁-FFK(Z₂)F (SEQ ID NO:55), X₁-ffk(Z₂)y or X₁-FFK(Z₂)Y (SEQ ID NO:56), X₁-ffyk(Z₂) or X₁-FFYK(Z₂) (SEQ ID NO:57), X₁-fffk(Z₂) or X₁-FFFK(Z₂) (SEQ ID NO:58), X₁-ffe(Z₃)-Z₁ or X₁-FFE(Z₃)-Z₁ (SEQ ID NO:59), X₁-fffe(Z₃)-Z₁ or X₁-FFFE(Z₃)-Z₁ (SEQ ID NO:60), X₁-ffk(Z₂)e(Z₃)-Z₁ or X₁-FFK(Z₂)E(Z₃)-Z₁ (SEQ ID NO:61), X₁-ffe(Z₃)k(Z₂)-Z₁ or X₁-FFE(Z₃)K(Z₂)-Z₁ (SEQ ID NO:62), X₁-ffffe(Z₃)-Z₁ or X₁-FFFFE(Z₃)-Z₁ (SEQ ID NO:63), X₁-ffe(Z₃)ff-Z₁ or X₁-FFE(Z₃)FF-Z₁ (SEQ ID NO:64), X₁-fffe(Z₃)f-Z₁ or X₁-FFFE(Z₃)F-Z₁ (SEQ ID NO:65), X₁-ffd(Z₃)-Z₁ or X₁-FFD(Z₃)-Z₁ (SEQ ID NO:66), X₁-fffd(Z₃)-Z₁ or X₁-FFFD(Z₃)-Z₁ (SEQ ID NO:67), X₁-ffk(Z₂)d(Z₃)-Z₁ or X₁-FFK(Z₂)D(Z₃)-Z₁ (SEQ ID NO:68), X₁-ffd(Z₃)k(Z₂)-Z₁ or X₁-FFD(Z₃)K(Z₂)-Z₁ (SEQ ID NO:69), X₁-ffffd(Z₃)-Z₁ or X₁-FFFFD(Z₃)-Z₁ (SEQ ID NO:70), X₁-ffd(Z₃)ff-Z₁ or X₁-FFD(Z₃)FF-Z₁ (SEQ ID NO:71), X₁-fffd(Z₃)f-Z₁ or X₁-FFFD(Z₃)F-Z₁ (SEQ ID NO:72), X₁-nal-fff-Z₁ or X₁-Nal-FFF-Z₁ (SEQ ID NO:73), X₁-nal-ffff-Z₁ or X₁-Nal-FFFF-Z₁ (SEQ ID NO:74), X₁-nal-ffkf-Z₁ or X₁-Nal-FFKF-Z₁ (SEQ ID NO:75), X₁-nal-ffky-Z₁ or X₁-Nal-FFKY-Z₁ (SEQ ID NO:76), X₁-nal-ffyk-Z₁ or X₁-Nal-FFYK-Z₁ (SEQ ID NO:77), X₁-nal-fffk-Z₁ or X₁-Nal-FFFK-Z₁ (SEQ ID NO:78), X₁-nal-fffff-Z₁ or X₁-Nal-FFFFF-Z₁ (SEQ ID NO:79), X₁-nal-ffgff-Z₁ or X₁-Nal-FFGFF-Z₁ (SEQ ID NO:80), X₁-nal-fffgf-Z₁ or X₁-Nal-FFFGF-Z₁ (SEQ ID NO:81), X₁-nal-ffffg-Z₁ or X₁-Nal-FFFFG-Z₁ (SEQ ID NO:82), X₁-nal-ffk(Z₂)f or X₁-Nal-FFK(Z₂)F (SEQ ID NO:83), X₁-nal-ffk(Z₂)y or X₁-Nal-FFK(Z₂)Y (SEQ ID NO:84), X₁-nal-ffyk(Z₂) or X₁-Nal-FFYK(Z₂) (SEQ ID NO:85), X₁-nal-fffk(Z₂) or X₁-Nal-FFFK(Z₂) (SEQ ID NO:86), X₁-nal-ffe(Z₃)-Z₁ or X₁-Nal-FFE(Z₃)-Z₁ (SEQ ID NO:87), X₁-nal-fffe(Z₃)-Z₁ or X₁-Nal-FFFE(Z₃)-Z₁ (SEQ ID NO:88), X₁-nal-ffk(Z₂)e(Z₃)-Z₁ or X₁-Nal-FFK(Z₂)E(Z₃)-Z₁ (SEQ ID NO:89), X₁-nal-ffe(Z₃)k(Z₂)-Z₁ or X₁-Nal-FFE(Z₃)K(Z₂)-Z₁ (SEQ ID NO:90), X₁-nal-ffffe(Z₃)-Z₁ or X₁-Nal-FFFFE(Z₃)-Z₁ (SEQ ID NO:91), X₁-nal-ffe(Z₃)ff-Z₁ or X₁-Nal-FFE(Z₃)FF-Z₁ (SEQ ID NO:92), X₁-nal-fffe(Z₃)f-Z₁ or X₁-Nal-FFFE(Z₃)F-Z₁ (SEQ ID NO:93), X₁-nal-ffd(Z₃)-Z₁ or X₁-Nal-FFD(Z₃)-Z₁ (SEQ ID NO:94), X₁-nal-fffd(Z₃)-Z₁ or X₁-Nal-FFFD(Z₃)-Z₁ (SEQ ID NO:95), X₁-nal-ffk(Z₂)d(Z₃)-Z₁ or X₁-Nal-FFK(Z₂)D(Z₃)-Z₁ (SEQ ID NO:96), X₁-nal-ffd(Z₃)k(Z₂)-Z₁ or X₁-Nal-FFD(Z₃)K(Z₂)-Z₁ (SEQ ID NO:97), X₁-nal-ffffd(Z₃)-Z₁ or X₁-Nal-FFFFD(Z₃)-Z₁ (SEQ ID NO:98), X₁-nal-ffd(Z₃)ff-Z₁ or X₁-Nal-FFD(Z₃)FF-Z₁ (SEQ ID NO:99), and X₁-nal-fffd(Z₃)f-Z₁ or X₁-Nal-FFFD(Z₃)F-Z₁ (SEQ ID NO:100),
wherein lowercase letters denote D-amino acids, uppercase letters denote L-amino acids, nal is a D-naphthylalanine (3-(I-Naphthyl)-D-alanine or 3-(2-Naphthyl)-D-alanine), and Nal is an L-naphthylalanine (3-(I-Naphthyl)-L-alanine or 3-(2-Naphthyl)-L-alanine);
X₁ is 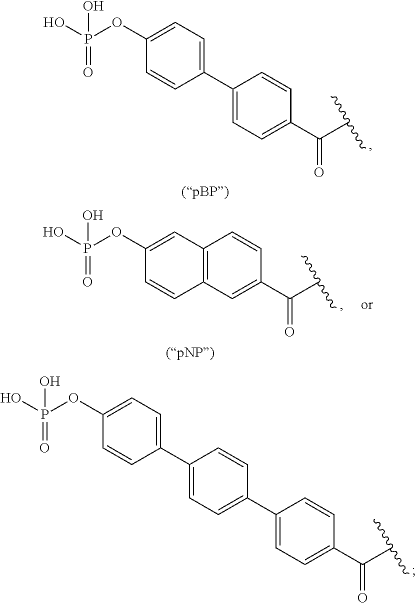 [SMILES: *C(=O)c1ccc(-c2ccc(OP(=O)(O)O)cc2)cc1 ; *C(=O)c1ccc2cc(OP(=O)(O)O)ccc2c1 ; *C(=O)c1ccc(-c2ccc(-c3ccc(OP(=O)(O)O)cc3)cc2)cc1]
Z₁ is selected from the group of-OH (the unmodified C-terminal amino acid residue), —O—CH₃ (methylation of the C-terminal amino acid residue), a fluorophore, a chemotherapeutic agent, an antiangiogenic agent, a thermoablative nanoparticle, an immunomodulating agent, or an antigen;
Z₂ is optional and is selected from the group of-H (the unmodified amino group on the lysine sidechain), a fluorophore, a chemotherapeutic agent, an antiangiogenic agent, a thermoablative nanoparticle, an immunomodulating agent, or an antigen; and
Z₃ is selected from the group of-OH (the unmodified acidic side chain) and —O—CH₃ (methylation of the acidic side chain).

16. A product formed by exposing the peptide of claim 1 to an enzyme that hydrolyzes the phosphate group.

17. The product according to claim 16, wherein the product is selected from the group of:
X₂-fff-Z₁ or X₂-FFF-Z₁, X₂-ffff-Z₁ or X₂-FFFF-Z₁ (SEQ ID NO:101), X₂-ffkf-Z₁ or X₂-FFKF-Z₁ (SEQ ID NO:102), X₂-ffky-Z₁ or X₂-FFKY-Z₁ (SEQ ID NO:103), X₂-ffyk-Z₁ or X₂-FFYK-Z₁ (SEQ ID NO:104), X₂-fffk-Z₁ or X₂-FFFK-Z₁ (SEQ ID NO:10⁵), X₂-fffff-Z₁ or X₂-FFFFF-Z₁ (SEQ ID NO:106), X₂-ffgff-Z₁ or X₂-FFGFF-Z₁ (SEQ ID NO:107), X₂-fffgf-Z₁ or X₂-FFFGF-Z₁ (SEQ ID NO:108), X₂-ffffg-Z₁ or X₂-FFFFG-Z₁ (SEQ ID NO:109), X₂-ffk(Z₂)f or X₂-FFK(Z₂)F (SEQ ID NO:110), X₁-ffk(Z₂)y or X₂-FFK(Z₂)Y (SEQ ID NO:111), X₂-ffyk(Z₂) or X₂-FFYK(Z₂) (SEQ ID NO:112), X₂-fffk(Z₂) or X₂-FFFK(Z₂) (SEQ ID NO:113), X₂-ffe(Z₃)-Z₁ or X₂-FFE(Z₃)-Z₁ (SEQ ID NO:114), X₂-fffe(Z₃)-Z₁ or X₂-FFFE(Z₃)-Z₁ (SEQ ID NO:115), X₂-ffk(Z₂)e(Z₃)-Z₁ or X₂-FFK(Z₂)E(Z₃)-Z₁ (SEQ ID NO:116), X₂-ffe(Z₃)k(Z₂)-Z₁ or X₂-FFE(Z₃)K(Z₂)-Z₁ (SEQ ID NO:117), X₂-ffffe(Z₃)-Z₁ or X₂-FFFFE(Z₃)-Z₁ (SEQ ID NO:118), X₂-ffe(Z₃)ff-Z₁ or X₂-FFE(Z₃)FF-Z₁ (SEQ ID NO:119), X₂-fffe(Z₃)f-Z₁ or X₂-FFFE(Z₃)F-Z₁ (SEQ ID NO:120), X₂-ffd(Z₃)-Z₁ or X₂-FFD(Z₃)-Z₁ (SEQ ID NO:121), X₂-fffd(Z₃)-Z₁ or X₂-FFFD(Z₃)-Z₁ (SEQ ID NO:122), X₂-ffk(Z₂)d(Z₃)-Z₁ or X₂-FFK(Z₂)D(Z₃)-Z₁ (SEQ ID NO:123), X₂-ffd(Z₃)k(Z₂)-Z₁ or X₂-FFD(Z₃)K(Z₂)-Z₁ (SEQ ID NO:124), X₂-ffffd(Z₃)-Z₁ or X₂-FFFFD(Z₃)-Z₁ (SEQ ID NO:125), X₂-ffd(Z₃)ff-Z₁ or X₂-FFD(Z₃)FF-Z₁ (SEQ ID NO:126), X₂-fffd(Z₃)f-Z₁ or X₂-FFFD(Z₃)F-Z₁ (SEQ ID NO:127), X₂-nal-fff-Z₁ or X₂-Nal-FFF-Z₁ (SEQ ID NO:128), X₂-nal-ffff-Z₁ or X₂-Nal-FFFF-Z₁ (SEQ ID NO:129), X₂-nal-ffkf-Z₁ or X₂-Nal-FFKF-Z₁ (SEQ ID NO:130), X₂-nal-ffky-Z₁ or X₂-Nal-FFKY-Z₁ (SEQ ID NO:131), X₂-nal-ffyk-Z₁ or X₂-Nal-FFYK-Z₁ (SEQ ID NO:132), X₂-nal-fffk-Z₁ or X₂-Nal-FFFK-Z₁ (SEQ ID NO:133), X₂-nal-fffff-Z₁ or X₂-Nal-FFFFF-Z₁ (SEQ ID NO:134), X₂-nal-ffgff-Z₁ or X₂-Nal-FFGFF-Z₁ (SEQ ID NO:135), X₂-nal-fffgf-Z₁ or X₂-Nal-FFFGF-Z₁ (SEQ ID NO:136), X₂-nal-ffffg-Z₁ or X₂-Nal-FFFFG-Z₁ (SEQ ID NO:137), X₂-nal-ffk(Z₂)f or X₁-Nal-FFK(Z₂)F (SEQ ID NO:138), X₂-nal-ffk(Z₂)y or X₁-Nal-FFK(Z₂)Y (SEQ ID NO:139), X₂-nal-ffyk(Z₂) or X₁-Nal-FFYK(Z₂) (SEQ ID NO:140), X₂-nal-fffk(Z₂) or X₁-Nal-FFFK(Z₂) (SEQ ID NO:141), X₂-nal-ffe(Z₃)-Z₁ or X₂-Nal-FFE(Z₃)-Z₁ (SEQ ID NO:142), X₂-nal-fffe(Z₃)-Z₁ or X₂-Nal-FFFE(Z₃)-Z₁ (SEQ ID NO:143), X₂-nal-ffk(Z₂)e(Z₃)-Z₁ or X₂-Nal-FFK(Z₂)E(Z₃)-Z₁ (SEQ ID NO:144), X₂-nal-ffe(Z₃)k(Z₂)-Z₁ or X₂-Nal-FFE(Z₃)K(Z₂)-Z₁ (SEQ ID NO:145), X₂-nal-ffffe(Z₃)-Z₁ or X₂-Nal-FFFFE(Z₃)-Z₁ (SEQ ID NO:146), X₂-nal-ffe(Z₃)ff-Z₁ or X₂-Nal-FFE(Z₃)FF-Z₁ (SEQ ID NO:147), X₂-nal-fffe(Z₃)f-Z₁ or X₂-Nal-FFFE(Z₃)F-Z₁ (SEQ ID NO:148), X₂-nal-ffd(Z₃)-Z₁ or X₂-Nal-FFD(Z₃)-Z₁ (SEQ ID NO:149), X₂-nal-fffd(Z₃)-Z₁ or X₂-Nal-FFFD(Z₃)-Z₁ (SEQ ID NO:150), X₂-nal-ffk(Z₂)d(Z₃)-Z₁ or X₂-Nal-FFK(Z₂)D(Z₃)-Z₁ (SEQ ID NO:151), X₂-nal-ffd(Z₃)k(Z₂)-Z₁ or X₂-Nal-FFD(Z₃)K(Z₂)-Z₁ (SEQ ID NO:152), X₂-nal-ffffd(Z₃)-Z₁ or X₂-Nal-FFFFD(Z₃)-Z₁ (SEQ ID NO:153), X₂-nal-ffd(Z₃)ff-Z₁ or X₂-Nal-FFD(Z₃)FF-Z₁ (SEQ ID NO:154), and X₂-nal-fffd(Z₃)f-Z₁ or X₂-Nal-FFFD(Z₃)F-Z₁ (SEQ ID NO:155);
wherein 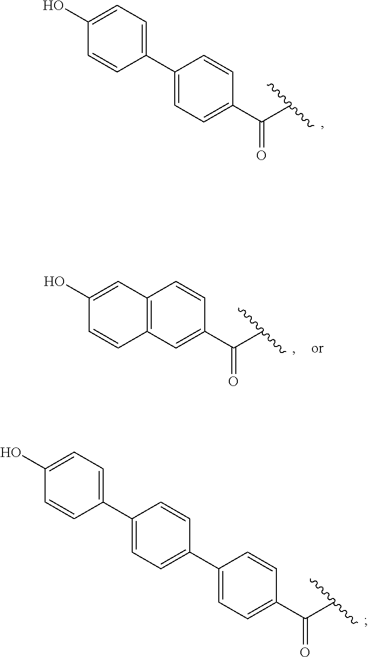 [SMILES: *C(=O)c1ccc(-c2ccc(O)cc2)cc1 ; *C(=O)c1ccc2cc(O)ccc2c1 ; *C(=O)c1ccc(-c2ccc(-c3ccc(O)cc3)cc2)cc1]
Z₁ is selected from the group of-OH (the unmodified C-terminal amino acid residue), —O—CH₃ (methylation of the C-terminal amino acid residue), a fluorophore, a chemotherapeutic agent, an antiangiogenic agent, a thermoablative nanoparticle, an immunomodulating agent, or an antigen;
Z₂ is optional and is selected from the group of-H (the unmodified amino group on the lysine sidechain), a fluorophore, a chemotherapeutic agent, an antiangiogenic agent, a thermoablative nanoparticle, an immunomodulating agent, or an antigen; and
Z₃ is selected from the group of-OH (the unmodified acidic side chain) and —O—CH₃ (methylation of the acidic side chain).

18. A supermolecular hydrogel formed upon self-assembly of the product of claim 16.

19. A supermolecular hydrogel formed upon self-assembly of a hydrolytic product of the peptide according to claim 1.

20. A pharmaceutical composition comprising a pharmaceutically acceptable carrier and a peptide according to claim 1.

21.-24. (canceled)

25. A method for forming a nanofibril network on or near the surface of target cells, the method comprising: contacting a target cell that expresses a cell surface-bound enzyme having hydrolytic activity, secretes an enzyme having hydrolytic activity, or both, with the peptide according to claim 1 or a pharmaceutical composition comprising said peptide, wherein said contacting is effective to hydrolyze the phosphate group and cause in situ self-assembly of the peptides to form a nanofibril network on or near the surface of the target cell.

26.-36. (canceled)

37. A method for collecting a target cell secretome comprising: contacting a target cell that expresses a cell surface-bound enzyme having hydrolytic activity, secretes an enzyme having hydrolytic activity, or both, with the peptide according to claim 1 or a pharmaceutical composition comprising said peptide, wherein said contacting is effective to hydrolyze the phosphate group and cause in situ self-assembly of the peptide to form a nanofibril network on or near the surface of the target cell, whereby the nanofibril network retains the target cell secretome from the pericellular space of the target cell; separating the target cell secretome from the nanofibril network; and collecting the separated target cell secretome.

38.-52. (canceled)

53. A method for treating a cancerous condition comprising: administering to a subject having a cancerous condition a therapeutically effective amount of the peptide according to claim 1 the or a pharmaceutical composition comprising said peptide, wherein said administering is effective to hydrolyze the phosphate group and cause in vivo self-assembly of the peptides to form a nanofibril network on or near the surface of cancer cells.

54.-71. (canceled)
